(19) United States
(12) Patent Application Publication
(10) Pub. No.: US 2024/0173332 A1
(43) Pub. Date: May 30, 2024

(54) BENZODIAZEPINES FOR TREATING OR PREVENTING RESPIRATORY SYNCYTIAL VIRUS INFECTION

(71) Applicant: Pfizer Inc., New York, NY (US)

(72) Inventors: Matthew Barrett, Sedgefield (GB); Alexandre Bedernjak, Sedgefield (GB); George Stuart Cockerill, Sedgefield (GB); James Good, Valencia (ES)

(73) Assignee: Pfizer Inc., New York, NY (US)

(21) Appl. No.: 18/547,244

(22) PCT Filed: Feb. 24, 2022
(86) PCT No.: PCT/GB2022/050504

§ 371 Date: Aug. 21, 2023

(30) Foreign Application Priority Data
Feb. 24, 2021 (GB) 2102602.6

(51) Int. Cl. A61K 31/5513; A61K 9/00; A61K 9/08; A61K 9/20; A61K 45/06; A61K 47/10; A61K 47/12; A61K 47/22; A61K 47/26; A61K 47/69; A61P 31/14; C07D 487/04

(52) U.S. Cl. CPC A61K 31/5513; A61K 9/0019; A61K 9/08; A61K 9/2009; A61K 9/2013; A61K 9/2018; A61K 9/2059; A61K 45/06; A61K 47/10; A61K 47/12; A61K 47/22; A61K 47/26; A61K 47/6951; A61P 31/14; C07D 487/04

(57) ABSTRACT

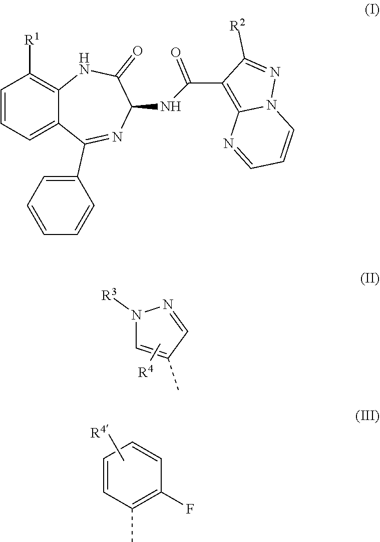

Pharmacuetical Compounds Benzodiazepine derivatives of formula (I): wherein: R¹ is H or F; R² is selected from formula (II) and formula (III) R³ is C₁-C₆ alkyl which is unsubstituted or substituted by CF₃, or is a monocyclic 4- to 6-membered heterocyclic group containing 1 or 2 heteroatoms selected from O, N and S; R⁴ is H or a group selected from —X, -alk-X, —CONR⁶R⁷, C═NNR⁶R⁷, —SO₂R⁶ and —SO₂NR⁶R⁷; R⁴ is a group selected from —X, -alk-X, —CONR⁶R⁷, —C═NNR⁶R⁷, —SO₂R⁶ and —SO₂R⁶R⁷; X is OH or a derivative of an OH group selected from α-amino carboxylic acid esters, carboxylic acid esters, carbonates, carbamates, ethers and phosphates: alk is C₁-C₆ alkylene which is unsubstituted or substituted by C₃-C₆ cycloalkyl; and R⁶ and R⁷ are each independently C₁-C₆ alkyl or C₃-C₆ cycloalkyl; and the pharmaceutically acceptable salts thereof are inhibitors of respiratory syncytial virus (RSV) and can therefore be used to treat or prevent an RSV infection.

### FIELD OF THE INVENTION

The present invention relates to benzodiazepine derivatives and prodrugs thereof, and to their use in treating or preventing a respiratory syncytial virus (RSV) infection.

### BACKGROUND TO THE INVENTION

RSV is a negative-sense, single-stranded RNA virus of the Paramyxoviridae family. RSV is readily transmitted by secretions from an infected person via surfaces or hand-to-hand transfer. Unlike influenza, it is not transmitted by small-particle aerosols. Following successful inoculation, the incubation period is between four and six days during which time the virus spreads from the nasopharynx to the lower respiratory tract by fusion of infected with uninfected cells and by sloughing of the necrotic epithelium. In infants, coupled with increased mucus secretion and oedema, this can lead to mucus plugging causing hyper-inflation and collapse of distal lung tissue indicative of bronchiolitis. Hypoxia is common and the ability to feed is often impaired because of respiratory distress. In RSV pneumonia, inflammatory infiltration of the airways consists of mononuclear cells and is more generalised, with involvement of the bronchioles, bronchi and alveoli. The duration and degree of viral shedding has been found to correlate with the clinical signs and severity of disease.

RSV is the leading cause of serious respiratory tract infections in infants and young children throughout the world. The highest morbidity and mortality occurs in those born prematurely and for those with chronic lung or heart disease, although many infants hospitalised for RSV infection are otherwise healthy. Severe RSV infection in infancy can lead to several years of recurrent wheezing and is linked to the later development of asthma.

RSV is also a major cause of morbidity and mortality in the elderly and in immunocompromised children and adults as well as those with chronic obstructive pulmonary disease (COPD) and congestive heart failure (CHF).

RSV has a seasonal incidence; it is highly predictable and occurs in the winters of both hemispheres, from September to May in Europe and North America, peaking in December and January, and can occur throughout the year in tropical countries. It affects >90% of infants and young children by the age of two years and as natural immunity is short-lived; many will be re-infected each year. As with influenza, in elderly people, RSV causes around 10% of winter hospitalisations with an associated mortality of 10%.

Current anti-RSV treatment involves the use of a monoclonal antibody to RSV, called palivizumab. Such use of palivizumab is a prophylactic, rather than therapeutic, treatment of RSV. Although this antibody is often effective, its use is restricted to preterm infants and infants at high risk. Indeed, its limited utility means that it is unavailable for many people in need of anti-RSV treatment. There is therefore an urgent need for effective alternatives to existing anti-RSV treatment.

Small molecules have also been proposed as inhibitors of RSV. These include benzimidazoles and benzodiazepines. For instance, the discovery and initial development of RSV604, a benzodiazepine compound having sub-micromolar anti-RSV activity, is described in Antimicrobial Agents and Chemotherapy, Sept. 2007, 3346-3353 (Chapman et al). Benzodiazepine inhibitors of RSV are also disclosed in publications including WO2004/026843 and WO2005/089770 (Arrow Therapeutics Limited); WO2016/166546 and WO2018/033714 (Durham University); and WO2017/015449, WO2018/129287 and WO2018/226801 (Enanta Pharmaceuticals, Inc.).

A prodrug strategy may be used to improve the aqueous solubility, permeability and/or chemical stability of a pharmaceutically active compound such as an inhibitor of RSV. The pharmacokinectic profile may also be improved through chemical masking of vulnerable functionalities within the active pharmaceutical compound. This includes, but is not limited to, increasing exposure, prolonging exposure or enabling delivery options. Enabling delivery options includes, for instance, switching from oral to parenteral delivery (i.e. administration by injection, infusion or implantation).

### SUMMARY OF THE INVENTION

It has now been found that a novel series of benzodiazepine compounds have potent anti-RSV activity with favourable pharmacokinetics and physicochemical properties. Some of the compounds possess an OH functional group which can be derivatised to form a prodrug. Such prodrugs are part of the present invention. Accordingly, the present invention provides a compound which is a benzodiazepine of formula (I):

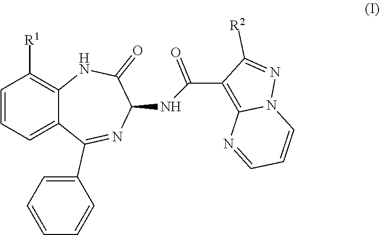

wherein:
R¹ is H or F;
R² is selected from:

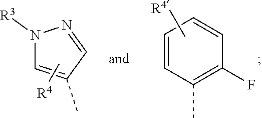

R³ is C₁-C₆ alkyl which is unsubstituted or substituted by CF₃, or is a monocyclic 4- to 6-membered heterocyclic group containing 1 or 2 heteroatoms selected from O, N and S;
R⁴ is H or a group selected from —X, -alk-X, —CONR⁶R⁷, C═NN R⁶R⁷, —SO₂R⁶ and —SO₂NR⁶R⁷;
R^{4′} is a group selected from —X, -alk-X, —CONR⁶R⁷, —C═NN R⁶R⁷, —SO₂R⁶ and —SO₂NR⁶R⁷;
X is —OH or a derivative of an OH group selected from α-amino carboxylic acid esters, carboxylic acid esters, carbonates, carbamates, ethers and phosphates;
alk is C₁-C₆ alkylene which is unsubstituted or substituted by C₃-C₆ cycloalkyl; and
R⁶ and Ware each independently C₁-C₆ alkyl or C₃-C₆ cycloalkyl;
or a pharmaceutically acceptable salt thereof.

Compounds of the invention possess two N atoms in the five-membered ring of the pyrazolopyrimidine ring that is linked via an amide group to the benzodiazepine ring system. This structural feature is thought to be important to the properties of the compounds discussed further below.

### DETAILED DESCRIPTION OF THE INVENTION

When any group, ring, substituent or moiety defined herein is substituted, it is typically substituted by Q as defined below.

A C₁₋₆ alkyl group or moiety is linear or branched. A C₁₋₆ alkyl group is typically a C₁₋₄ alkyl group, or a C₄₋₆ alkyl group. Examples of C₁₋₆ alkyl groups and moieties include methyl, ethyl, n-propyl, propyl, n-butyl, i-butyl, t-butyl, n-pentyl, i-pentyl (i.e. 3-methylbut-1-yl), t-pentyl (i.e. 2-methylbut-2-yl), neopentyl (i.e. 2,2-dimethylpropan-1-yl), n-hexyl, i-hexyl (i.e. 4-methylpentan-1-yl), t-hexyl (i.e. 3-methylpentan-3-yl) and neopentyl (i.e. 3,3-dimethylbutan-1-yl). For the avoidance of doubt, where two alkyl moieties are present in a group, the alkyl moieties may be the same or different. A C₁₋₆ alkyl group is unsubstituted or substituted , typically by one or more groups Q as defined below. For example, a C₁₋₆ alkyl group is unsubstituted or substituted by 1, 2 or 3 groups Q as defined below.

A C₁₋₆ alkylene group or moiety is a divalent, unsubstituted or substituted, linear or branched, saturated divalent aliphatic hydrocarbon group or moiety containing 1 to 6 carbon atoms. Typically it is a C₁₋₃ alkylene group or moiety. Examples include —CH₂—, —CH₂CH₂—, —CH(CH₃)—, —CH₂CH(CH₃)—, —CH₂C(CH₃)₂— and —C(CH₃)₂—. When C₁₋₆ alkylene is substituted by cycloalkyl, it is substituted on any constituent carbon atom by 1 or 2 cycloalkyl groups, typically one cycloalkyl group. Typically a C₁₆ alkylene moiety is unsubstituted or substituted by one cyclopropyl group.

Q is halo, nitro, —CN, OH, C₁₋₆ alkoxy, C₁₋₆ hydroxyalkyl, C₁₋₆ alkoxyalkyl, unsubstituted C₁₋₆ alkyl, C₁₋₆ alkylthio, C₁₋₆ haloalkyl, C₁₋₄ haloalkoxy, —CO₂R″′, —NR′₂, —SR′, —S(═O)R′, —S(═O)₂R′, C₃-C₁₀ cycloalkyl, 4- to 10-membered heterocyclyl, C₆-C₁₀ aryl or 4- to 10-membered heteroaryl, wherein each R′ is independently selected from H, C₁₋₆ alkyl, C₃-io cycloalkyl, 4 to 10-membered heterocyclyl, C₆-C₁₀ aryl and 4- to 10-membered heteroaryl.

A C₁₋₆ alkoxy group is linear or branched. It is typically a C₁₋₄ alkoxy group, for example a methoxy, ethoxy, propoxy, i-propoxy, n-propoxy, n-butoxy, sec-butoxy or tert-butoxy group. A C₁₆ alkoxy group is unsubstituted or substituted, typically by one or more groups Q as defined above.

A C₁₋₆ alkylthio group is linear or branched. It is typically a C₁₋₄ alkylthio group, for example a methylthio, ethylthio, propylthio, i-propylthio, n-propylthio, n-butylthio, sec-butylthio or tert-butylthio group. A C₁₋₆ alkylthio group is unsubstituted or substituted, typically by one or more groups Q as defined above.

A halogen or halo group is F, Cl, Br or I. Typically it is F or Cl. A C₁₋₆ alkyl group substituted by halogen may be denoted “C₁₋₆ haloalkyl”, which means a C₁₋₆ alkyl group as defined above in which one or more hydrogens is replaced by halo. Likewise a C₁₋₆ alkoxy group substituted by halogen may be denoted “C₁₋₆ haloalkoxy”, which means a C₁₋₆ alkoxy group as defined above in which one or more hydrogens is replaced by halo. Typically, C₁₋₆ haloalkyl or C₁₋₆ haloalkoxy is substituted by 1, 2 or 3 said halogen atoms. Haloalkyl and haloalkoxy groups include perhaloalkyl and perhaloalkoxy groups such as —CZ3 and —OCZ3 wherein Z is a halogen, for example —CF₃—CCl₃—OCF₃ and —OCCl₃.

A C₁₋₆ hydroxyalkyl group is a C₁₋₆ alkyl group as defined above, substituted by one or more OH groups. Typically, it is substituted by one, two or three OH groups. Preferably, it is substituted by a single OH group.

A C₁₋₆ alkoxyalkyl group is a C₁₋₆ alkyl group as defined above, substituted by a C₁₋₆ alkoxy group as defined above. It may, for instance, be methoxyalkyl or ethoxyalkyl, in which the alkyl moiety is a C₁₋₆ alkyl group as defined above

A C₆-C₁₀ aryl group is an aromatic carbocyclic group containing from 6 to 10 carbon atoms. It is monocyclic or a fused bicyclic ring system in which an aromatic ring is fused to another aromatic carbocyclic ring. Examples of a C₆-C₁₀ aryl group include phenyl and naphthyl. When substituted, an aryl group is typically substituted by a group Q as defined above, for instance by 1, 2 or 3, groups selected from a a group Q as defined above. More particularly, a substituted aryl group such as a substituted phenyl group is substituted by 1 or 2 groups selected from C₁-C₆ alkyl, halo, —OR— and —N(R⁸)₂ wherein R⁸ is H or C₁-C₆ alkyl, each R⁸ being the same or different when two are present.

A C₃₋₁₀ cycloalkyl group is a saturated hydrocarbon ring having from 3 to 10 carbon atoms. A C₃₋₁₀ cycloalkyl group may be, for instance, C₃-C₇ cycloalkyl such as cyclopropyl, cyclobutyl, cyclopentyl, cyclohexyl, or cycloheptyl. Typically it is C₃-C₆ cycloalkyl, or C₄-C₆ cycloalkyl, for example cyclobutyl, cyclopentyl or cyclohexyl. In one embodiment it is cyclobutyl. A C₃₋₁₀ cycloalkyl group is unsubstituted or substituted, typically by one or more groups Q as defined above.

A 4- to 10-membered heteroaryl group or moiety is a 4- to 10-membered aromatic heterocyclic group which contains 1, 2, 3, or 4 heteroatoms selected from O, N and S. It is monocyclic or bicyclic. Typically it contains one N atom and 0, 1, 2 or 3 additional heteroatoms selected from O, S and N. It may be, for example, a monocyclic 5- to 7-membered heteroaryl group, for instance a 5- or 6-membered N-containing heteroaryl group. Examples include pyridyl, pyrazinyl, pyrimidinyl, pyridazinyl, furanyl, thienyl, pyrazolidinyl, pyrrolyl, oxadiazolyl, oxazolyl, isoxazolyl, thiazolyl, thiadiazolyl, imidazolyl and pyrazolyl groups. Furanyl, thienyl, imidazolyl, pyridyl and pyrimidyl groups are preferred. It may alternatively be a bicyclic heteroaryl group, for instance an 8- to 10-membered bicyclic heteroaryl group. Examples include quinolyl, isoquinolyl, quinazolyl, quinoxalinyl, indolyl, isoindolyl, indazolyl, imidazopyridazinyl, pyrrolopyridinyl, pyrazolopyrimidinyl and pyrrolopyrimidinyl. When substituted, a heteroaryl group (monocyclic or bicyclic) is typically substituted by one or more, e.g. 1, 2 or 3, groups selected from C₁₋₄ alkyl and a group Q as defined above.

A 4- to 10-membered heterocyclyl group is a monocyclic or bicyclic non-aromatic, saturated or unsaturated ring system containing 5 to 10 carbon atoms and at least one atom or group selected from N, O, S, SO, SO₂ and CO, more typically N or O. When the ring system is bicyclic, one ring may be saturated and one ring unsaturated. Typically, it is a C₄-10 ring system in which 1, 2 or 3 of the carbon atoms in the ring are replaced with an atom or group selected from O, S, SO₂, CO and NH. More typically it is a monocyclic ring, preferably a monocyclic C₄-C₆ ring. Examples of a 4- to 10-membered heterocyclyl group include azetidinyl, piperidyl, piperazinyl, morpholinyl, thiomorpholinyl, S,S-dioxothiomorpholinyl, 1,3-dioxolanyl, pyrrolidinyl, imidazol-2-onyl, pyrrolidin-2-onyl, tetrahydrofuranyl and tetrahydropyranyl, piperidin-2,6-dionyl and piperidin-2-onyl moieties. In particular, a 4- to 10-membered heterocyclyl group may be azetidinyl, piperidyl, piperazinyl, morpholinyl or thiomorpholinyl

More particularly, a monocyclic 4- to 6-membered heterocyclic group containing 1 or 2 heteroatoms selected from O, N and S is typically a C₄-6 ring system in which 1 or 2 of the carbon atoms in the ring are replaced with an atom or group selected from O, S, or NH. Examples of a 4- to 6-membered heterocyclyl group include azetidinyl, piperidyl, piperazinyl, morpholinyl, thiomorpholinyl, S,S-dioxothiomorpholinyl, 1,3-dioxolanyl, pyrrolidinyl, imidazol-2-onyl, pyrrolidin-2-onyl, tetrahydrofuranyl and tetrahydropyranyl, piperidin-2,6-dionyl and piperidin-2-onyl moieties. In particular, a 4- to 6-membered heterocyclyl group is tetrahydropyranyl.

When substituted, a heterocyclic group (monocyclic or bicyclic) is typically substituted by one or more, e.g. 1, 2 or 3, groups selected from unsubstituted C₁₋₄ alkyl and a group Q as defined above. It may also be substituted by a bridgehead atom which links two of the ring atoms, typically two ring carbon atoms. For instance, a piperazine group or a morpholine group may be substituted by a carbon bridgehead. The resulting bicyclic structure may be, respectively, a 2,5-diazabicyclo[2.2.1]heptane or 2-oxa-5-azabicyclo[2.2.1]heptane group.

For the avoidance of doubt, although the above definitions of heteroaryl and heterocyclyl groups refer to an “N” atom which can be present in the ring, it will be evident to a skilled chemist that any such N atom will be protonated (or will carry a substituent as defined above) if it is attached to each of its adjacent ring atoms via a single bond. Such protonated forms are embraced within the present definitions of heteroaryl and heterocyclyl groups.

Formula (I) as defined above encompasses prodrugs. In general, suitable functional groups that allow an active inhibitor to serve as the anti-RSV moiety for a prodrug strategy include hydroxyl, amino and carboxylic acid groups. In compounds of present formula (I), the functional group that is exploited in this context is a hydroxyl group (—OH). The hydroxyl group acts as a handle for the introduction of prodrug moieties (or “promoieties”), which derivatise the —OH function to form prodrug compounds within formula (I).

Suitable prodrug strategies that allow attachment of prodrug components to a hydroxyl group include carboxylic acid esters, carbonates, carbamates, ethers and phosphates, for instance as described by Rautio et al (Nature Reviews Drug Discovery, 2008, 7, 255-270). The incorporation of a prodrug moiety can lead to recognition by endogenous transport systems, leading to active transport. For example, a common strategy is to prodrug hydroxylic groups as α-amino carboxylic acid esters.

The prodrugs within present formula (I) as defined above are the compounds in which X is defined as a derivative of an —OH group selected from α-amino carboxylic acid esters, carboxylic acid esters, carbonates, carbamates, ethers and phosphates.

An α-amino carboxylic acid consists of an amino group (—NH₂) and a carboxyl group (—COOH) attached to a central C atom to which an organic side-chain (R) is also attached. The central C atom is also referred to as the α-C atom. The structure is:

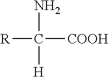

The side-chain R is characteristic of the individual amino acid in question. In the 20 common amino acids shown in the table below, R is the group attached to the NH₂-bearing C atom. In the case of glycine, R is H. In the case of proline, R is a 5-membered ring incorporating the N atom of the amino group.

TABLE 1
Structures of common amino acids

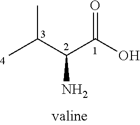

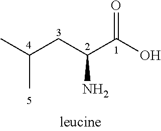

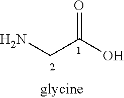

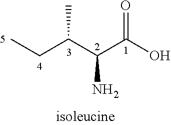

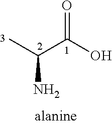

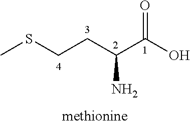

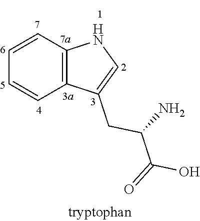 [SMILES: N[C@@H](Cc1c[nH]c2ccccc12)C(=O)O]

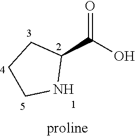

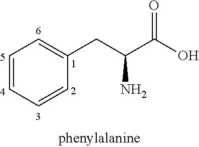 [SMILES: N[C@@H](Cc1ccccc1)C(=O)O]

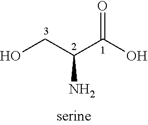

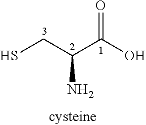

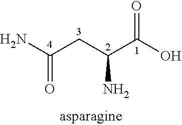

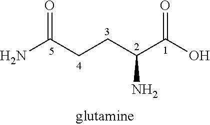

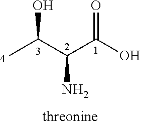

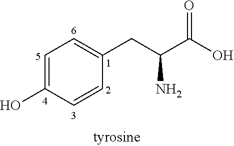 [SMILES: N[C@@H](Cc1ccc(O)cc1)C(=O)O]

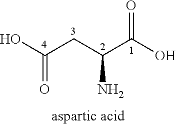

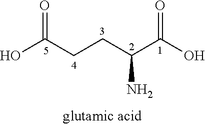

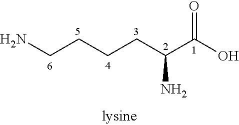

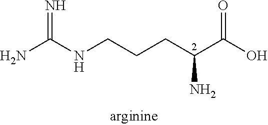 [SMILES: N=C(N)NCCC[C@H](N)C(=O)O]

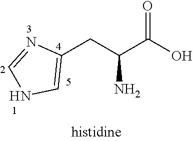 [SMILES: N[C@@H](Cc1c[nH]cn1)C(=O)O]

The carboxy function of an α-amino carboxylic acid may undergo esterification with an OH-containing compound to form an α-amino carboxylic acid ester. Thus, when in present formula (I) group X is an α-amino carboxylic acid ester derivative of —OH, X may be a group —OC(O)—CHR—NH₂ in which R is the side chain of an α-amino carboxylic acid (typically a common amino acid, as shown in Table 1 above).

In compounds of present formula (I) of this type, the H attached to the α-C atom, and/or to one or both of the amino group H atoms, may be replaced by methyl. It is also possible for the amino group to form an amide linkage with the carboxy function of a second α-amino carboxylic acid, thereby forming a dipeptide grouping. When group X in present formula (I) is an α-amino carboxylic acid ester derivative of —OH, it is therefore typically defined as a group —OC(O)—C(R¹⁰)(Y)—NR¹¹₂ in which R¹⁰ is independently H or Me, each R¹¹ is independently H, Me or a group —C(O)—CHY—NR¹⁰₂ and Y is the side-chain of an α-amino carboxylic acid (or common amino acid). In one embodiment, R¹⁰ is H, each R¹¹ is H, and Y is selected from H, methyl, isopropyl and sec-butyl.

Carboxylic acid ester derivatives of —OH are formed by esterification with the carboxy function of a carboxylic acid. The carboxylic acid may be of formula R⁹COOH, wherein R⁹ is C₁-C₆ alkyl which is unsubstituted or substituted. The resulting derivative group is then of formula —OC(O)R⁹

Carbonate derivatives of —OH are formed by esterification with the carboxy function of a carbonic acid The carbonic acid may be of formula R⁹ OCOOH, wherein R⁹ is as defined above. The resulting derivative group is then of formula —OC(O)OR⁹.

Carbamate derivatives of —OH are formed by esterification with the carboxy function of carbamic acid or an N-substituted derivative thereof. The carbamic acid may be of formula NR¹²₂COOH in which each R¹² is independently H or C₁-C₆ alkyl which is unsubstituted or substituted. The resulting derivative group is then of formula —OCONR¹²₂.

Ether derivatives of —OH are formed by etherification using standard techniques, such as treatment of the deprotonated —OH group with an organic halide or reductive etherification. The resulting derivative group is of formula —OR⁹ in which R⁹ is as defined above.

Phosphate derivatives of —OH are typically phosphate monoester derivatives, optionally including a methylene bridging group, formed by phosphorylation or other standard techniques. The resulting derivative group is of formula —OP(O)(OH)₂ or —OCH₂—OP(O)(OH)₂. For instance, the phosphate derivative group may be of formula —OP(O)(OH)₂.

In compounds of formula (I), R⁴ is H or a group selected from —X, -alk-X, —CONR⁶R⁷, C═NNR⁶R⁷, —SO₂R⁶ and —SO₂NR⁶R⁷ in which X, alk, R⁶ and R⁷ are as defined above. R^{4′} is a group selected from —X, -alk-X, —CONR⁶R⁷, C═NNR⁶R⁷, —SO₂R⁶ and —SO₂NR⁶R⁷ in which X, alk, R⁶ and R⁷ are as defined above. Accordingly, in both R⁴ and R^{4′}, X is —OH or a derivative of an OH group selected from α-amino carboxylic acid esters, carboxylic acid esters, carbonates, carbamates, ethers and phosphates, for instance a derivative of an —OH group selected from those described above.

In one embodiment of formula (I), X is —OH. In another embodiment X is —OH or a derivative of —OH selected from phosphates and α-amino carboxylic acid esters.

In a further embodiment of formula (I), X is —OR⁸ in which R⁸ is H or a group selected from —C(O)—C(R¹⁰)(y)—NR¹¹₂, —C(O)R⁹, —C(O)OR⁹, —C(O)NR¹²₂, —CH₂—OP(O)(OH)₂ and —P(O)(OH)₂, wherein:
each R⁹ is independently C₁-C₆ alkyl which is unsubstituted or substituted;
each R¹⁰ is independently H or Me;
each R¹¹ is independently H, Me or a group —C(O)—CHY—NR¹⁰₂,
each R¹² is independently H or C₁-C₆ alkyl which is unsubstituted or substituted; and
Y is the side-chain of an α-amino carboxylic acid, such as a common amino acid as defined above.

In both R⁴ and R^{4′}, alk is C₁-C₆ alkylene which is unsubstituted or substituted by C₃-C₆ cycloalkyl. In one embodiment of formula (I), alk is selected from —CH₂, —CH₂CH₂—, —CH(CH₃)—, —C(CH₃)₂—, —CH₂CH(CH₃)—, —CH₂C(CH₃)₂—and cyclopropyl-substituted alkylene groups of formulae:

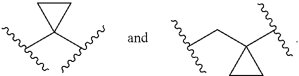 [SMILES: *C1(*)CC1 ; *CC1(*)CC1]

In one embodiment of formula (I), R⁴ is H.

In compounds of formula (I), R³ is C₁-C₆ alkyl which is unsubstituted or substituted by CF₃, or is a monocyclic 4- to 6-membered heterocyclic group containing 1 or 2 heteroatoms selected from O, N and S. In one embodiment R³ is C₁-C₆ alkyl which is unsubstituted or substituted by CF_{3,} or is a tetrayhdropyranyl group. In another embodiment R³ is selected from isopropyl, —CH₂CF₃ and tetrahydropyranyl. In a further embodiment R³ is selected from isopropyl, ethyl, —CH₂CF₃ and tetrahydropyranyl.

Specific compounds of the invention include the following:
2-[2-Fluoro-4-(hydroxymethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-[2-Fluoro-4-(hydroxymethyl)phenyl]-N-[(3S)-2-oxo-5-phenyl-1,3-dihydro-1,4-benzodiazepin-3-yl]pyrazolo[1,5-a]pyrimidine-3-carboxamide;
N-[(3S)-9-fluoro-2-oxo-5-phenyl-1,3-dihydro-1,4-benzodiazepin-3-yl]-2-[1-(2,2,2-trifluoroethyl)pyrazol-4-yl]pyrazolo[1,5-a]pyrimidine-3-carboxamide;
N-[(3S)-9-fluoro-2-oxo-5-phenyl-1,3-dihydro-1,4-benzodiazepin-3-yl]-2-(1-propan-2-ylpyrazol-4-yl)pyrazolo[1,5-a]pyrimidine-3-carboxamide;
N-[(3S)-2-oxo-5-phenyl-1,3-dihydro-1,4-benzodiazepin-3-yl]-2-(1-propan-2-ylpyrazol-4-yl)pyrazolo[1,5-a]pyrimidine-3-carboxamide;
N-[(3S)-9-fluoro-2-oxo-5-phenyl-1,3-dihydro-1,4-benzodiazepin-3-yl]-2-[1-(oxan-4-yl)pyrazol-4-yl]pyrazolo[1,5-a]pyrimidine-3-carboxamide;
2-(2-Fluoro-4-methylsulfonylphenyl)-N-[(3S)-9-fluoro-2-oxo-5-phenyl-1,3-dihydro-1,4-benzodiazepin-3-yl]pyrazolo[1,5-a]pyrimidine-3-carboxamide,
2-(2-Fluoro-4-methylsulfonylphenyl)-N-[(3S)-2-oxo-5-phenyl-1,3-dihydro-1,4-benzodiazepin-3-yl]pyrazolo[1,5-a]pyrimidine-3-carboxamide;
2-[2-Fluoro-5-(hydroxymethyl)phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-5-(hydroxymethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-(2-Fluoro-4-hydroxyphenyl)-N-[(3S)-2-oxo-5-phenyl-1,3-dihydro-1,4-benzodiazepin-3-yl]pyrazolo[1,5-a]pyrimidine-3-carboxamide;
2-(2-Fluoro-4-hydroxyphenyl)-N-[(3S)-9-fluoro-2-oxo-5-phenyl-1,3-dihydro-1,4-benzodiazepin-3-yl]pyrazolo[1,5-a]pyrimidine-3-carboxamide;
2-(1-Ethylpyrazol-4-yl)-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-4-(2-hydroxy-2-methylpropyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-[2-Fluoro-4-(2-hydroxy-2-methylpropyl)phenyl]-N-[(3S)-2-oxo-5-phenyl-1,3-dihydro-1,4-benzodiazepin-3-yl]pyrazolo[1,5-a]pyrimidine-3-carboxamide,
2-[2-Fluoro-4-(1-hydroxyethyl)phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-4-(1-hydroxyethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-[2-Fluoro-3-(hydroxymethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-[2-Fluoro-4-(2-hydroxypropan-2-yl)phenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-[2-Fluoro-4-(2-hydroxypropan-2-yl)phenyl]-N-[(3S)-2-oxo-5-phenyl-1,3-dihydro-1,4-benzodiazepin-3-yl]pyrazolo[1,5-a]pyrimidine-3-carboxamide,
2-[2-Fluoro-4-(2-hydroxyethyl)phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-4-(2-hydroxyethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-(4-Ethylsulfonyl-2-fluorophenyl)-N-[(3S)-9-fluoro-2-oxo-5-phenyl-1,3-dihydro-1,4-benzodiazepin-3-yl]pyrazolo[1,5-a]pyrimidine-3-carboxamide,
N-[(3S)-9-Fluoro-2-oxo-5-phenyl-1,3-dihydro-1,4-benzodiazepin-3-yl]-2-(2-fluoro-4-sulfamoylphenyl)pyrazolo[1,5-a]pyrimidine-3-carboxamide;
2-[4-(Dimethylsulfamoyl)-2-fluorophenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-(2-Fluoro-5-methylsulfonylphenyl)-N-[(3S)-9-fluoro-2-oxo-5-phenyl-1,3-dihydro-1,4-benzodiazepin-3-yl]pyrazolo[1,5-a]pyrimidine-3-carboxamide,
2-(4-Carbamoyl-2-fluorophenyl)-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-4-[(1S*)-1-hydroxyethyl]phenyl]-N-[(3S)-2-oxo-5-phenyl-1,3-dihydro-1,4-benzodiazepin-3-yl]pyrazolo[1,5-a]pyrimidine-3-carboxamide,
2-[2-Fluoro-4-[(1R*)-1-hydroxyethyl]phenyl]-N-[(3S)-2-oxo-5-phenyl-1,3-dihydro-1,4-benzodiazepin-3-yl]pyrazolo[1,5-a]pyrimidine-3-carboxamide,
2-[2-Fluoro-4-[(1S*)-1-hydroxyethyl]phenyl]-N-[(3S)-9-fluoro-2-oxo-5-phenyl-1,3-dihydro-1,4-benzodiazepin-3-yl]pyrazolo[1,5-a]pyrimidine-3-carboxamide,
2-[2-Fluoro-4-[(1R*)-1-hydroxyethyl]phenyl]-N-[(3S)-9-fluoro-2-oxo-5-phenyl-1,3-dihydro-1,4-benzodiazepin-3-yl]pyrazolo[1,5-a]pyrimidine-3-carboxamide,
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl aminoacetate hydrochloride;
3-Fluoro-4-(3-{[(3S)-2-oxo-5-phenyl-1,3-dihydro-1,4-benzodiazepin-3-yl]carbamoyl}pyrazolo[1,5-a]pyrimidin-2-yl)phenyl]methyl aminoacetate hydrochloride;
[3-Fluoro-4-(3-{[(3S)-2-oxo-5-phenyl-1,3-dihydro-1,4-benzodiazepin-3-yl]carbamoyl}pyrazolo[1,5-a]pyrimidin-2-yl)phenyl]methyl (2S)-2-amino-3-methylbutanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2S)-2-aminopropanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2R)-2-aminopropanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2S)-2-amino-3-methylbutanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2R)-2-amino-3-methylbutanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2S)-2-amino-4-methylpentanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2R)-2-amino-4-methylpentanoate hydrochloride;
[3-Fluoro-4-[3-[[(3S)-9-fluoro-2-oxo-5-phenyl-1,3-dihydro-1,4-benzodiazepin-3-yl]carbamoyl]pyrazolo[1,5-a]pyrimidin-2-yl]phenyl]methyl dihydrogen phosphate; and the pharmaceutically acceptable salts thereof.

The compounds of the invention may contain asymmetric or chiral centres, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Compounds of Formula (I) containing one or more chiral centre may be used in enantiomerically or diastereoisomerically pure form, or in the form of a mixture of isomers.

The present invention embraces all geometric and positional isomers of compounds of the invention as defined above. For example, if a compound of the invention incorporates a double bond or a fused ring, the cis- and trans-forms, as well as mixtures thereof, are embraced within the scope of the invention. Both the single positional isomers and mixture of positional isomers are also within the scope of the present invention.

The compounds of the present invention may exist in unsolvated as well as solvated forms with pharmaceutically acceptable solvents such as water, ethanol, and the like, and it is intended that the invention embrace both solvated and unsolvated forms.

The compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol tautomerizations. Valence tautomers include interconversions by reorganization of some of the bonding electrons.

Compounds of the invention can be prepared by the synthetic methods described in the Examples that follow, or by analogy with such methods using appropriate starting materials and methodologies familiar to the skilled chemist.

Compounds of the invention containing prodrug moieties, i.e. compounds in which X is a derivative of an OH group as defined in formula (I) above, can be prepared by known methods for preparing prodrugs. Methods of preparing prodrugs are known in the literature, for example as described by Subbaiah et al. (J. Med. Chem. 2019, 62, 7, 3553-3574). For instance, carboxylic ester prodrugs containing an amino acid promoiety (compounds of formula (I) in which X is an α-amino carboxylic acid ester derivative of OH) may be prepared from a hydroxyl containing parent compound and an N-Boc protected amino acid. Methods to perform this include esterification with N,N′-dicyclohexylcarbodiimide in combination with 4-dimethylaminopyridine, and subsequent removal of the N-Boc protecting group by acidic hydrolysis with a strong acid such as hydrochloric acid.

Methods for preparing phosphate ester prodrugs (compounds of formula (I) in which X is a phosphate derivative of OH) include preparing phosphate monoester prodrugs from a hydroxyl containing parent compound, for instance as summarised by Domon et al., ACS Central Science. 2020, 6, 2, 283-292. The methods described include phosphorylation of a hydroxyl functional group with a pentavalent phosphoryl donor such as phosphoryl chloride, or use of a trivalent phosphoramidite reagent with subsequent oxidation to yield a protected phosphate triester. This may be deprotected to the phosphate monoester by removal of the protecting groups with suitable conditions dependent on the nature of the protecting group and the sensitivity of the molecule to acidic, basic or reductive deprotection conditions.

A benzodiazepine derivative of formula (I) can be converted into a pharmaceutically acceptable salt thereof, and a salt can be converted into the free compound, by conventional methods. For instance, a benzodiazepine derivative of formula (I) can be contacted with a pharmaceutically acceptable acid to form a pharmaceutically acceptable salt. A pharmaceutically acceptable salt is a salt with a pharmaceutically acceptable acid or base.

Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic orp-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

Compounds of the present invention have been found in biological tests to be inhibitors of respiratory syncytial virus (RSV). They possess a combination of potent anti-RSV activity with favourable bioavailability and physicochemical characteristics. This combination of properties makes the compounds therapeutically useful and superior as drug candidates to many compounds disclosed in the prior art references discussed earlier.

Accordingly, the present invention further provides a compound which is a benzodiazepine derivative of formula (I), as defined above, or a pharmaceutically acceptable salt thereof, for use in a method of treating the human or animal body by therapy.

The invention also provides a compound of the invention as defined above for use in a method treating or preventing an RSV infection. Still further, the present invention provides the use of a compound of the invention as defined above in the manufacture of a medicament for use in treating or preventing an RSV infection. A subject suffering from or susceptible to an RSV infection may thus be treated by a method comprising the administration thereto of a compound of the invention as defined above. The condition of the subject may thereby be improved or ameliorated.

The RSV infection is typically a respiratory tract infection. The RSV infection may be an infection in a child, for instance a child under ten years of age or an infant under two years of age. In one embodiment the invention provides a compound as defined above for use in treating or preventing an RSV infection in paediatric patients. Alternatively the infection may be an infection in a mature or elderly adult, for instance an adult over 60 years of age, an adult over 70 years of age, or an adult over 80 years of age. The invention further provides a compound for use in treating or preventing an RSV infection in geriatric patients.

The RSV infection may be an infection in an immunocompromised individual or an individual suffering from COPD or CHF. In another embodiment, the RSV infection is an infection in a non-compromised individual, for instance an individual who is otherwise healthy.

A compound of the present invention can be administered in a variety of dosage forms, for example orally such as in the form of tablets, capsules, sugar- or film-coated tablets, liquid solutions or suspensions or parenterally, for example intramuscularly, intravenously or subcutaneously. The compound may therefore be given by injection, infusion, or by inhalation or nebulisation. The compound is preferably given by oral administration.

The dosage depends on a variety of factors including the age, weight and condition of the patient and the route of administration. Daily dosages can vary within wide limits and will be adjusted to the individual requirements in each particular. Typically, however, the dosage adopted for each route of administration when a compound is administered alone to adult humans is 0.0001 to 650 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule.

A unit dose form such as a tablet or a capsule will usually contain 1-250 mg of active ingredient. For example, a compound of formula (I) could be administered to a human patient at a dose of between 100-250 mg either once a day, twice or three times a day. For example, a compound of formula (I) could be administered to a human patient at a dose of between 100-250 mg either once a day, twice or three times a day.

The compounds of formula (I) and pharmaceutically acceptable salts thereof may be used on their own. Alternatively, they may be administered in the form of a pharmaceutical composition. The present invention therefore also provides a pharmaceutical composition comprising a compound of formula (I) or a pharmaceutically acceptable salt thereof as hereinbefore defined, in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The compounds of the invention may be administered in a variety of dosage forms. Thus, they can be administered orally, for example as tablets, troches, lozenges, aqueous or oily suspensions, solutions, dispersible powders or granules. The compounds of the invention may also be administered parenterally, whether subcutaneously, intravenously, intramuscularly, intrasternally, transdermally, by infusion techniques or by inhalation or nebulisation. The compounds may also be administered as suppositories.

Solid oral forms of the pharmaceutical composition of the invention may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulfates; and, in general, non-toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride. Further suitable carriers for suspensions include sterile water, hydroxypropylmethyl cellulose (HPMC), polysorbate 80, polyvinylpyrrolidone (PVP), aerosol AOT (i.e. sodium 1,2-bis(2-ethylhexoxycarbonyl)ethanesulphonate), pluronic F127 and/or captisol (i.e. sulfobutylether-beta-cyclodextrin).

The compounds of the invention may, for example, be formulated as aqueous suspensions in a carrier selected from:
(i) 0.5% w/v hydroxypropylmethyl cellulose (HPMC)/0.1% w/v polysorbate 80;
(ii) 0.67% w/v polyvinylpyrrolidone (PVP)/0.33% w/v aerosol AOT (sodium 1,2-bis(2-ethylhexoxycarbonyl)ethanesulphonate);
(iii) 1% w/v pluronic F 127; and
(iv) 0.5% w/v polysorbate 80.

The carriers may be prepared by standard procedures known to those of skill in the art. For example, each of the carriers (i) to (iv) may be prepared by weighing the required amount of excipient into a suitable vessel, adding approximately 80% of the final volume of water and magnetically stirring until a solution is formed. The carrier is then made up to volume with water. The aqueous suspensions of compounds of formula I may be prepared by weighing the required amount of a compound of formula I into a suitable vessel, adding 100% of the required volume of carrier and magnetically stirring.

Solutions for injection or infusion may contain as carrier, for example, sterile water or preferably they may be in the form of sterile, aqueous, isotonic saline solutions.

The compounds of the invention may also be administered in conjunction with other compounds used for the treatment of viral infections. Thus, the invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment or prevention of a viral infection, particularly infection by RSV.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

Suitable therapeutic agents for use in the combination therapies include
(i) RSV fusion inhibitors
(ii) other RSV nucleocapsid (N)-protein inhibitors
(iii) other RSV protein inhibitors, such as those that inhibit the phosphoprotein (P) protein and large (L) protein;
(iv) nucleoside or polymerase inhibitors that inhibit the L Protein;
(v) anti-RSV monoclonal antibodies, such as the F-protein antibodies;
(vi) immunomodulating toll-like receptor compounds;
(vii) other respiratory virus anti-virals, such as anti-influenza and anti-rhinovirus compounds; and/or
(viii) anti-inflammatory compounds.

The RSV nucleocapsid (N)-protein plays a pivotal role in viral transcription and replication, mediating the interaction between the genomic RNA and the virally encoded RNA-dependent RNA polymerase. The RSV P- and L-proteins are components of RSV's virally encoded RNA-dependent RNA polymerase.

According to a further aspect of the invention, there is provided a compound of the formula (I) or a pharmaceutically acceptable salt thereof as hereinbefore defined in combination with one or more of the therapeutic agents listed as (i) to (vi) above for use in the treatment of RSV.

The Examples that follow serve to illustrate the invention further. The Preparatory Examples relate to the preparation of starting materials and intermediates used to prepare the compounds of the Examples. Neither the Examples nor the Preparatory Examples limit the invention in any way.

### EXAMPLES

Reagents were obtained from commercial sources and were used without further purification. Anhydrous solvents were purchased from commercial suppliers, used as supplied and stored under N₂. Reactions were performed with anhydrous solvents under an atmosphere of N₂ unless otherwise noted. All temperatures are in ° C. TLC was performed on aluminium backed silica gel plates with fluorescence indicator at 254 nM (median pore size 60 Å). Flash column chromatography was performed using a Biotage Isolera One system using KP-Sil, Ultra, Sfar Duo or Sfax HC silica gel columns.

NMR spectra were recorded on a 400, 600 or 700 MHz spectrometer at ambient probe temperature (nominal 298 K). Chemical shifts (δ) are given in ppm and calibrated by using the residual peak of the solvent as the internal standard (CDCl₃, δ=7.26 ppm; DMSO-d₆, δ=2.50 ppm). Coupling constants are given in Hertz (Hz). LRMS were recorded using an Advion Plate Express expression^{L} compact mass spectrometer equipped with an APCI ion source.

LCMS analysis was performed by the following methods. LCMS Method A: Acquity BEH C18 column (2.1×50 mm; 1.7 μm), column temperature 60° C., flow rate 1.00 mL/min, photodiode array (PDA) detector wavelength 220-300 nm, with a linear gradient 0-100% over 2 minutes. Mobile phase A: water (0.1% v/v TFA). Mobile phase B: acetonitrile. LCMS Method B: Acquity BEH C18 column (2.1×150 mm; 1.7 μm), column temperature 60° C., flow rate 0.3 mL/min, PDA detector wavelength 220-300 nm, with a linear gradient 2-100% over 22.5 minutes. Mobile phase A: water (0.1% v/v NH₃). Mobile phase B: acetonitrile.

Mass spectra were recorded using a Waters Acquity QDa detector with ES switching between positive and negative ion mode.

Preparative HPLC was performed at ambient column temperature by the following methods. HPLC Method 1: Luna P6 (21.2 mm×150 mm, 5 μm), column temperature: ambient, flow rate 21 mL/min, PDA detector wavelength 210 nm. HPLC Method 2: Gemini NX C₁₈ (30 mm×150 mm, 5 μm), column temperature: ambient, flow rate 42 mL/min, PDA detector wavelength 240 nm.

Preparative SFC was performed by the following methods. SFC Method 1: Lux C₂ (21.2 mm×250 mm, 5 μm), column temperature 40° C., flow rate 50 mL/min, 100 BarG pressure, detector wavelength 229 nm. Analytical chiral SFC was performed by the following methods. SFC method 1A: Lux C2 (4.6 mm×250 mm, 5 μm), column temperature 40° C., flow rate 4 mL/min, detector wavelength 210-400 nm, 125 barG back pressure. SFC method 2A: Lux C4 (4.6 mm×250 mm, 5 μm), column temperature 40° C., flow rate 4 mL/min, detector wavelength 210-400 nm, 125 barG back pressure.

Preparatory examples 2-(4-ethylsulfonyl-2-fluorophenyl)-4,4,5,5-tetramethyl-1,3,2-dioxaborolane, 2-(2-fluoro-5-methylsulfonylphenyl)-4,4,5,5-tetramethyl-1,3,2-dioxaborolane, 3-fluoro-4-(4,4,5,5-tetramethyl-1,3,2-dioxaborolan-2-yl)benzenesulfonamide and 3-fluoro-N,N-dimethyl-4-(4,4,5,5-tetramethyl-1,3,2-dioxaborolan-2-yl)b enzenesulfonamide were prepared using methods described in WO/2022/008911.

Preparatory examples (3S)-3-amino-5-phenyl-1,3-dihydro-1,4-benzodiazepin-2-one and (3S)-3-amino-9-fluoro-5-phenyl-1,3-dihydro-1,4-benzodiazepin-2-one were prepared using methods described in WO/2004/026843, WO/2005/090319, and WO/2017/015449.

Abbreviations

| | |
|---|---|
| APCI | Atmospheric pressure chemical ionization |
| dppf | 1,1′-Bis(diphenylphosphino)ferrocene |
| DIPEA | N,N-Diisopropylethylamine |
| DMF | N,N-Dimethylformamide |
| DMSO | Dimethyl sulfoxide |
| H | Hour(s) |
| HATU | 1-[Bis(dimethylamino)methylene]-1H-1,2,3-triazolo[4,5-b] |
| | pyridinium 3-oxid hexafluorophosphate, N-[(dimethylamino)- |
| | 1H-1,2,3-triazolo-[4,5-b]pyridin-1-ylmethylene]-N- |
| | methylmethanaminium hexafluorophosphate N-oxide |
| LCMS | Liquid chromatography-mass spectrometry |
| LRMS | Low resolution mass spectrometry |
| MWI | Microwave irradiation |
| PDA | Photodiode array |
| rt | room temperature |
| SFC | Supercritical fluid chromatography |
| THF | Tetrahydrofuran |
| XPhos | 2-Dicyclohexylphosphino-2′,4′,6′-triisopropylbiphenyl |

### PREPARATORY EXAMPLES

### 1A Ethyl-5-amino-3-bromo-1H-pyrazole-4-carboxylate

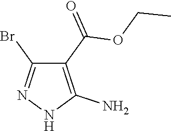

A solution of N-bromosuccinimide (13.77 g, 77.34 mmol) in MeCN (270 mL) was added dropwise over 25 min to a cooled (0° C.) solution of 5-amino-1H-pyrazole-4-carboxylic acid ethyl ester (10.00 g, 64.45 mmol) in THF (250 mL). The reaction was allowed to attain rt and stirred overnight. The reaction mixture was adsorbed onto silica gel, the volatiles removed under reduced pressure and the residue purified by column chromatography [10-50% (EtOH:CH₂Cl₂:NH₄OH; 50:8:1) in CH₂Cl₂] to afford a beige solid, which was triturated with CH₂Cl₂ (−20 mL) to afford a white solid (5.93 g, 39%). ¹H NMR (400 MHz, DMSO-d₆) δ 12.16 (s, 1H), 6.25 (s, 2H), 4.18 (q, J=7.1 Hz, 2H), 1.25 (t, J=7.1 Hz, 3H). LRMS (APCI+) m/z 234.1/236.1 [M+H]⁺.

### 2A Ethyl 2-bromopyrazolo[1,5-a]pyrimidine-3-carboxylate

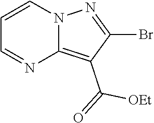 [SMILES: CCOC(=O)c1c(Br)nn2cccnc12]

1,1,3,3-Tetramethoxypropane (2.7 mL, 16.41 mmol) was added to a solution of intermediate 1A (2.4 g, 10.25 mmol) in acetic acid (35 mL) and heated at 70° C. for 18 h. The reaction mixture was cooled to rt and the volatiles removed under reduced pressure. The residue was suspended in water (20 mL), sat. aq. NaHCO₃ added (35 mL), and stirred at rt for 20 min. The suspension was filtered, washing with water followed by Et₂O (2×20 mL). The precipitate was dissolved in CH₂Cl₂, the volatiles removed under reduced pressure, and the residue purified by flash chromatography [10-15% (EtOH:CH₂Cl₂:NH₄OH; 50:8:1) in CH₂Cl₂]. The resultant solid was triturated with Et₂O (3×10 mL) and dried under reduced pressure to afford an off-white solid (1.79 g, 65%). ¹H NMR (400 MHz, DMSO-d₆) δ 9.24 (dd, J=7.0, 1.8 Hz, 1H), 8.85 (dd, J=4.2, 1.8 Hz, 1H), 7.33 (dd, J=7.0, 4.2 Hz, 1H), 4.32 (q, J=7.1 Hz, 2H), 1.32 (t, J=7.1 Hz, 3H). LRMS (APCI+) m/z 269.9, 271.9 [M+H]⁺.

### 3A Ethyl 2-11-(2,2,2-trifluoroethyl)pyrazol-4-ylkyrazolo[1,5-a]pyrimidine-3-carboxylate

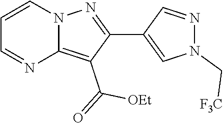 [SMILES: CCOC(=O)c1c(-c2cnn(CC(F)(F)F)c2)nn2cccnc12]

A mixture of intermediate 2A (207 mg, 0.77 mmol), 4-(4,4,5,5-tetramethyl-1,3,2-dioxaborolan-2-yl)-1-(2,2,2-trifluoroethyl)-1H-pyrazole (317 mg, 1.15 mmol) and K₂CO₃ (318 mg, 2.30 mmol) in 1,4-dioxane:water (2:1; 3.75 mL) was sparged with N₂ for 10 min. Pd(PPh₃)₄ (114 mg, 0.099 mmol) was added, the reaction mixture further degassed with N₂ for 3 min, then heated to 100° C. for 23 h. The reaction was cooled to rt, filtered through a pad of Celite® on glass microfiber filter paper, washing with CH₂Cl₂. The filtrate was washed with brine (5 mL), dried (MgSO₄) and the solvent removed under reduced pressure. Purification by column chromatography (40-100% EtOAc in heptane) afforded a yellow solid (258 mg, 99%). ¹H NMR (400 MHz, DMSO-d₆) δ 9.24 (dd, J=6.9, 1.7 Hz, 1H), 8.80 (dd, J=4.2, 1.7 Hz, 1H), 8.69 (s, 1H), 8.21 (s, 1H), 7.66-7.51 (m, 5H), 7.29 (dd, J=7.0, 4.2 Hz, 1H), 5.34-5.24 (m, 2H), 4.36 (q, J=7.1 Hz, 2H), 1.33 (t, J=7.1 Hz, 3H). LRMS (APCI+) m/z 340.0 [M+H]⁺

### 4A Ethyl 2-12-fluoro-4-(hydroxymethyl)phenylkyrazolo[1,5-a]pyrimidine-3-carboxylate

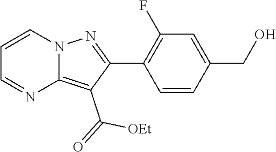 [SMILES: CCOC(=O)c1c(-c2ccc(CO)cc2F)nn2cccnc12]

Intermediate 2A (200 mg, 0.74 mmol), [2-fluoro-4-(hydroxymethyl)phenyl]boronic acid (189 mg, 1.11 mmol) and XPhos Pd G2 (29 mg, 0.037 mmol) were combined in a microwave vial, which was sealed, evacuated and sparged with N₂. A solution of 2 M aq. K₃PO₄ (0.74 mL, 1.48 mmol) and THF (2.22 mL), which had both been sparged with N_{2,} were added and the vial heated by MWI at 80° C. for 1 h. The cooled reaction mixture was diluted with EtOAc (10 mL), washed with brine (5 mL), dried (MgSO₄) and the solvent removed under reduced pressure. Purification by column chromatography (25-100% EtOAc in heptane) afforded a pale yellow solid (163 mg, 70%). ¹H NMR (400 MHz, DMSO-d₆) δ 9.32 (dd, J=7.0, 1.8 Hz, 1H), 8.85 (dd, J=4.2 1.7 Hz, 1H), 7.55 (t, J=7.6 Hz, 1H), 7.33 (dd, J=7.0, 4.2 Hz, 1H), 7.29-7.20 (m, 2H), 5.44 (t, J=5.8 Hz, 1H), 4.60 (d, J=5.8 Hz, 2H), 4.18 (q, J=7.1 Hz, 2H), 1.14 (t, J=7.1 Hz, 3H). LRMS (APCI+) m/z 338.1 [M+Na]⁺.

The following intermediate compounds were prepared in an analogous manner to intermediate 4A from the corresponding boronic acid pinacol ester. Intermediates 4E and 4I were prepared from the corresponding boronic acid. Intermediate 4G was prepared with heating at 100° C. by MWI for 1 h. Intermediates 4J to 4M were prepared with conventional heating at 85° C. overnight.

TABLE 1
Preparatory examples prepared via general Suzuki-Miyaura procedure.

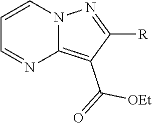

| | | | | LRMS m/z |
|---|---|---|---|---|
| Preparatory | | | ¹H NMR & | (APCI+); |
| Example | Name | R | (400 MHz) | TLC R_{f} |
| 4B | Ethyl 2-[1- (oxan-4- yl)pyrazol-4- yl]pyrazolo[1,5- a]pyrimidine-3- carboxylate | 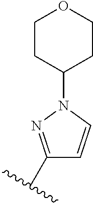 [SMILES: *c1ccn(C2CCOCC2)n1] | (DMSO-d₆) δ 9.22 (dd, J = 7.0, 1.7 Hz, 1H), 8.78 (dd, J = 4.2, 1.7 Hz, 1H), 8.54 (s, 1H), 8.11 (s, 1H), 7.26 (dd, J = 6.9, 4.2 Hz, 1H), 4.59-4.46 (m, 1H), 4.35 (q, J = 7.1 Hz, 2H), 4.04-3.92 (m, 2H), 3.54-3.41 (m, 2H), 2.08- 1.89 (m, 5H), 1.33 (t, J = 7.1 Hz, 3H). | 342.1 [M + H]⁺ |
| 4C | Ethyl 2-(1- propan-2- ylpyrazol-4- yl)pyrazolo[1,5- a]pyrimidine-3- carboxylate | 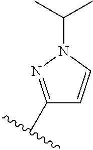 [SMILES: *c1ccn(C(C)C)n1] | (DMSO-d₆) δ 9.21 (dd, J = 6.9, 1.8 Hz, 1H), 8.78 (dd, J = 4.2, 1.8 Hz, 1H), 8.51 (d, J = 0.7 Hz, 1H), 8.09 (d, J = 0.7 Hz, 1H), 7.26 (dd, J = 6.9, 4.2 Hz, 1H), 4.61 (p, J = 6.7 Hz, 1H), 4.35 (q, J = 7.1 Hz, 2H), 1.47 (d, J = 6.7 Hz, 6H), 1.33 (t, J = 7.1 Hz, 3H). | 299.8 [M + H]⁺ |
| 4D | Ethyl 2-(2- fluoro-4- methylsulfonyl- phenyl)pyrazolo [1,5- a]pyrimidine-3- carboxylate | 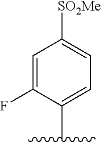 [SMILES: *c1ccc(S(C)(=O)=O)cc1F] | (DMSO-d₆) δ 9.36 (dd, J = 7.0, 1.7 Hz, 1H), 8.94-8.87 (m, 1H), 8.00-7.85 (m, 3H), 7.39 (dd, J = 7.0, 4.2 Hz, 1H), 4.19 (q, J = 7.1 Hz, 2H), 3.37 (s, 3H), 1.14 (t, J = 7.1 Hz, 3H). | 363.6 [M + H]⁺ |
| 4E | Ethyl 2-[2- fluoro-5- (hydroxymethyl) phenyl]pyrazolo [1,5- a]pyrimidine-3- carboxylate | 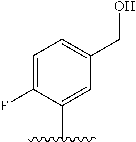 [SMILES: *c1cc(CO)ccc1F] | (DMSO-d₆) δ 9.31 (dd, J = 7.0, 1.7 Hz, 1H), 8.86 (dd, J = 4.2, 1.7 Hz, 1H), 7.54 (dd, J = 7.0, 2.3 Hz, 1H), 7.47 (ddd, J = 8.4, 5.1, 2.3 Hz, 1H), 7.34 (dd, J = 7.0, 4.2 Hz, 1H), 7.27 (dd, J = 10.1, 8.4 Hz, 1H), 5.33 (t, J = 5.8 Hz, 1H), 4.55 (d, J = 5.7 Hz, 2H), 4.17 (q, J = | 316.3 [M + H]⁺ |
| | | | 7.1 Hz, 2H), 1.13 (t, J = 7.1 Hz, | |
| | | | 3H). | |
| 4F | Ethyl 2-(2- fluoro-4- hydroxyphenyl) pyrazolo[1,5- a]pyrimidine-3- carboxylate | 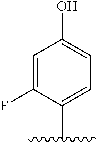 [SMILES: *c1ccc(O)cc1F] | (DMSO-d₆) δ 10.24 (s, 1H), 9.27 (dd, J = 7.0, 1.8 Hz, 1H), 8.82 (dd, J = 4.2, 1.8 Hz, 1H), 7.40 (t, J = 8.5 Hz, 1H), 7.29 (dd, J = 7.0, 4.2 Hz, 1H), 6.76- 6.62 (m, 2H), 4.18 (q, J = 7.1 Hz, 2H), 1.15 (t, J = 7.1 Hz, 3H). | 302.1 [M + H]⁺ |
| 4G | Ethyl 2-(1- ethylpyrazol-4- yl)pyrazolo[1,5- a]pyrimidine-3- carboxylate | 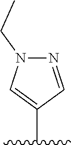 [SMILES: *c1cnn(CC)c1] | (DMSO-d₆) δ 9.21 (dd, J = 6.9, 1.8 Hz, 1H), 8.77 (dd, J = 4.2, 1.8 Hz, 1H), 8.50 (d, J = 0.6 Hz, 1H), 8.08 (d, J = 0.7 Hz, 1H), 7.26 (dd, J = 6.9, 4.2 Hz, 1H), 4.35 (q, J = 7.1 Hz, 2H), 4.23 (q, J = 7.3 Hz, 2H), 1.42 (t, J = 7.3 Hz, 3H), 1.33 (t, J = 7.1 Hz, 3H). | 286.2 [M + H]⁺ |
| 4H | Ethyl 2-[2- fluoro-3- (hydroxymethyl) phenyl]pyrazolo [1,5- a]pyrimidine-3- carboxylate | 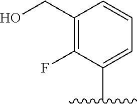 [SMILES: *c1cccc(CO)c1F] | (DMSO-d₆) δ 9.31 (dd, J = 7.0, 1.8 Hz, 1H), 8.86 (dd, J = 4.2, 1.7 Hz, 1H), 7.64-7.57 (m, 1H), 7.46 (dd, J = 7.0, 1.8 Hz, 1H), 7.35-7.28 (m, 2H), 5.38 (t, J = 5.7 Hz, 1H), 4.61 (d, J = 5.5 Hz, 2H), 4.15 | 316.3 [M + H]⁺ |
| | | | (q, J = 7.1 Hz, 2H), 1.10 (t, J = | |
| | | | 7.1 Hz, 3H). | |
| 4I | Ethyl 2-(4- carbamoyl-2- fluorophenyl) pyrazolo[1,5- a]pyrimidine-3- carboxylate | 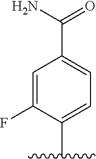 [SMILES: *c1ccc(C(N)=O)cc1F] | (DMSO-d₆) δ 9.34 (dd, J = 7.0, 1.7 Hz, 1H), 8.88 (dd, J = 4.2, 1.8 Hz, 1H), 8.18 (s, 1H), 7.84 (dd, J = 7.9, 1.6 Hz, 1H), 7.79 (dd, J = 11.0, 1.6 Hz, 1H), 7.69 (t, J = 7.5 Hz, 1H), 7.64 (s, 1H), 7.36 (dd, J = 7.0, 4.2 Hz, 1H), 4.18 (q, J = 7.1 Hz, 2H), 1.13 (t, J = 7.1 Hz, 3H). | 329.3 [M + H]⁺ |
| 4J | Ethyl 2-(4- ethylsulfonyl-2- fluorophenyl) pyrazolo[1,5- a]pyrimidine-3- carboxylate | 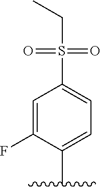 [SMILES: *c1ccc(S(=O)(=O)CC)cc1F] | (CDCl₃) δ 8.85 (dd, J = 4.4, 2.0 Hz, 1H), 8.78 (dd, J = 6.8, 1.6 Hz, 1H), 7.82-7.80 (m, 2H), 7.75-7.72 (m, 1H), 7.11 (dd, J = 7.2, 4.4 Hz, 1H), 4.33 (q, J = 7.2 Hz, 2H), 3.18 (q, J = 7.6 Hz, 2H), 1.34 (t, J = 7.6, 3H), 1.22 (t, J = 7.2 Hz, 3H) | R_{f} = 0.38 (EtOAc) |
| 4K | Ethyl 2-(2- fluoro-4- sulfamoylphenyl) pyrazolo[1,5- a]pyrimidine-3- carboxylate | 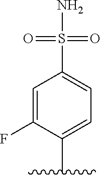 [SMILES: *c1ccc(S(N)(=O)=O)cc1F] | (CDCl₃) δ 9.35 (dd, J = 7.2, 2.0 Hz, 1H), 8.89 (dd, J = 4.0, 1.6 Hz, 1H), 7.84-7.77 (m, 2H), 7.73 (dd, J = 9.6, 1.6 Hz, 1H), 7.64 (s, 2H), 7.37 (dd, J = 7.2, 4.4 Hz, 1H), 4.20 (q, J = 7.2 Hz, 2H), 1.15 (t, J = 7.2 Hz, 3H). | R_{f} = 0.34 (EtOAc) |
| 4L | Ethyl 2-[4- (dimethylsulf- amoyl)-2- fluorophenyl] pyrazolo[1,5- a]pyrimidine-3- carboxylate | 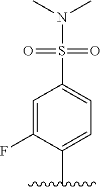 [SMILES: *c1ccc(S(=O)(=O)N(C)C)cc1F] | (CDCl₃) δ 8.88 (dd, J = 4.1, 1.8 Hz, 1H), 8.80 (dd, J = 7.0, 1.8 Hz, 1H), 7.80 (dd, J = 8.0, 6.5 Hz, 1H), 7.71 (dd, J = 8.0, 1.7 Hz, 1H), 7.63 (dd, J = 9.0, 1.7 Hz, 1H), 7.13 (dd, J = 7.0, 4.1 Hz, 1H), 4.35 (q, J = 7.1 Hz, 2H), 2.81 (s, 6H), 1.33- 1.19 (m, 3H). | R_{f} = 0.30 (EtOAc) |
| 4M | Ethyl 2-(2- fluoro-5- methylsulfonyl- phenyl)pyrazolo [1,5- a]pyrimidine-3- carboxylate | 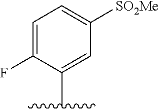 [SMILES: *c1cc(S(C)(=O)=O)ccc1F] | (CDCl₃) δ 8.22 (dd, J = 6.4, 2.8 Hz, 1H), 8.07 (ddd, J = 8.8, 4.4, 2.4 Hz, 1H), 7.37 (t, J = 8.8 Hz, 1H), 7.11 (dd, J = 7.2, 4.4 Hz, 1H), 7.33 (q, J = 7.2 Hz, 2H), 3.12 (s, 3H), 1.23 (t, J = 6.8 Hz, 3H). | R_{f} = 0.29 (EtOAc) |

### 5A 2-[2-Fluoro-4-(hydroxymethyl)phenyl]pyrazolo[1,5-a]pyrimidine-3-carboxylic acid

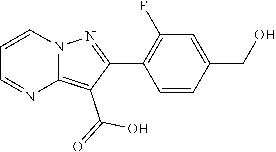 [SMILES: O=C(O)c1c(-c2ccc(CO)cc2F)nn2cccnc12]

A solution of intermediate 4A (160 mg, 0.51 mmol) and LiOH (1 M aq., 1.52 mL, 1.52 mmol) in THF/MeOH (3:1; 2.53 mL) was heated at 50° C. overnight. The volatiles were removed under reduced pressure, the residue was acidified with 1 M aq. HCl (1.66 mL) and extracted with CHCl₃/iPrOH (3:1; 3×15 mL). The combined organic extracts were washed with brine (5 mL), dried (MgSO₄) and the solvent removed under reduced pressure. The crude residue was triturated with ice-cold Et₂O (3×) and dried under reduced pressure to afford a pink solid (133 mg, 91%). ¹H NMR (600 MHz, DMSO-d₆) δ 12.28 (br s, 1H), 9.28 (dd, J=6.9, 1.7 Hz, 1H), 8.82 (dd, J=4.2, 1.8 Hz, 1H), 7.52 (t, J=7.6 Hz, 1H), 7.30 (dd, J=7.0, 4.1 Hz, 1H), 7.27-7.20 (m, 2H), 5.40 (br s, 1H), 4.59 (s, 2H). TLC R_{f} (200:8:1 CH₂Cl₂:EtOH:HCOOH)=0.125.

The following intermediate compounds were prepared by an analogous procedure to that described for intermediate 5A. Intermediates 5I to 5L were prepared with stirring at rt overnight.

TABLE 2
Preparatory Examples prepared via general ester hydrolysis procedure

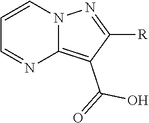

| Preparatory | | | ¹H NMR δ | LRMS m/z |
|---|---|---|---|---|
| Example | Name | R | (DMSO-d₆) | (APCI+) |
| 5B | 2-(1-Propan-2- ylpyrazol-4- yl)pyrazolo[1,5- a]pyrimidine-3- carboxylic acid | 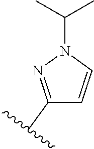 [SMILES: *c1ccn(C(C)C)n1] | (400 MHz) δ 12.39 (br s, 1H), 9.20 (dd, J = 6.9, 1.7 Hz, 1H), 8.74 (dd, J = 4.2, 1.7 Hz, 1H), 8.54 (d, J = 0.6 Hz, 1H), 8.13 (d, J = 0.6 Hz, 1H), 7.24 (dd, J = 6.9, 4.2 Hz, 1H), 4.60 (hept, J = 6.7 Hz, 1H), 1.46 (d, J = 6.7 Hz, 6H). | 271.8 [M + H]⁺ |
| 5C | 2-[1-(2,2,2- Trifluoroethyl) pyrazol-4- yl]pyrazolo[1,5- a]pyrimidine-3- carboxylic acid | 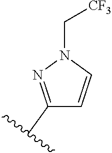 [SMILES: *c1ccn(CC(F)(F)F)n1] | (600 MHz) δ 12.44 (br s, 1H), 9.22 (dd, J = 6.9, 1.8 Hz, 1H), 8.77 (dd, J = 4.2, 1.7 Hz, 1H), 8.71 (s, 1H), 8.23 (d, J = 0.5 Hz, 1H), 7.26 (dd, J = 6.9, 4.2 Hz, 1H), 5.32-5.25 (m, 2H). | 311.8 [M + H]⁺ |
| 5D | 2-[1-(Oxan-4- yl)pyrazol-4- yl]pyrazolo[1,5- a]pyrimidine-3- carboxylic acid | 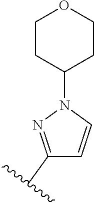 [SMILES: *c1ccn(C2CCOCC2)n1] | (600 MHz) δ 12.40 (br s, 1H), 9.20 (dd, J = 6.9, 1.7 Hz, 1H), 8.74 (dd, J = 4.1, 1.7 Hz, 1H), 8.57 (s, 1H), 8.15 (s, 1H), 7.24 (dd, J = 6.9, 4.2 Hz, 1H), 4.56-4.47 (m, 1H), 4.01- 3.93 (m, 2H), 3.52- 3.44 (m, 2H), 2.06-1.91 (m, 4H). | 313.8 [M + H]⁺ |
| 5E | 2-(2-Fluoro-4- methylsulfonyl- phenyl)pyrazolo[1,5- a]pyrimidine-3- carboxylic acid | 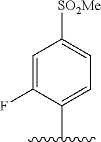 [SMILES: *c1ccc(S(C)(=O)=O)cc1F] | (400 MHz) δ 12.44 (br s, 1H), 9.33 (dd, J = 7.0, 1.8 Hz, 1H), 8.87 (dd, J = 4.1, 1.9 Hz, 1H), 7.98- 7.85 (m, 3H), 7.36 (dd, J = 6.9, 4.1 Hz, 1H), 3.39 (s, 3H). | 352.9 [M + NH₄]⁺ |
| 5F | 2-[2-Fluoro-5- (hydroxymethyl) phenyl]pyrazolo[1,5- a]pyrimidine-3- carboxylic acid | 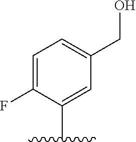 [SMILES: *c1cc(CO)ccc1F] | (400 MHz) δ 12.24 (s, 1H), 9.28 (dd, J = 7.0, 1.8 Hz, 1H), 8.82 (dd, J = 4.2, 1.8 Hz, 1H), 7.51 (dd, J = 7.0, 2.3 Hz, 1H), 7.49-7.40 (m, 1H), 7.36- 7.21 (m, 2H), 4.54 (s, 2H). | 326.1 [M + K]⁺ |
| 5G | 2-(1-Ethylpyrazol- 4-yl)pyrazolo[1,5- a]pyrimidine-3- carboxylic acid | 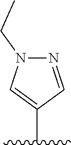 [SMILES: *c1cnn(CC)c1] | (400 MHz) δ 12.36 (s, 1H), 9.20 (dd, J = 6.9, 1.8 Hz, 1H), 8.74 (dd, J = 4.2, 1.7 Hz, 1H), 8.53 (s, 1H), 8.12 (s, 1H), 7.24 (dd, J = 6.9, 4.2 Hz, 1H), 4.22 (q, J = 7.3 Hz, 2H), 1.41 (t, J= 7.3 Hz, 3H). | 258.0 [M + H]⁺ |
| 5H | 2-[2-Fluoro-3- (hydroxymethyl)ph enyl]pyrazolo[1,5- a]pyrimidine-3- carboxylic acid | 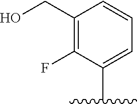 [SMILES: *c1cccc(CO)c1F] | (400 MHz) δ 12.28 (s, 1H), 9.28 (dd, J = 7.0, 1.7 Hz, 1H), 8.82 (dd, J = 4.2, 1.7 Hz, 1H), 7.62- 7.56 (m, 1H), 7.48-7.42 (m, 1H), 7.33-7.26 (m, 2H), 5.37 (t, J = 5.8 Hz, | 287.9 [M + H]⁺ |
| | | | 1H), 4.60 (d, J = 5.7 Hz, | |
| | | | 2H). | |
| 5I | 2-(4-Ethylsulfonyl- 2- fluorophenyl) pyrazolo[1,5- a]pyrimidine-3- carboxylic acid | 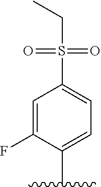 [SMILES: *c1ccc(S(=O)(=O)CC)cc1F] | (400 MHz) δ 9.33 (dd, J = 7.2, 2.0 Hz, 1H), 8.87 (dd, J = 4.4, 2.0 Hz, 1H), 7.91-7.84 (m, 3H), 7.35 (dd, J = 6.8, 4.0 Hz, 1H), 3.46 (q, J = 7.2 Hz, 2H), 1.17 (t, J = 7.2 Hz, 3H). | Not available |
| 5J | 2-(2-Fluoro-4- sulfamoylphenyl) pyrazolo[1,5- a]pyrimidine-3- carboxylic acid | 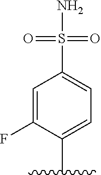 [SMILES: *c1ccc(S(N)(=O)=O)cc1F] | (400 MHz) δ 12.45 (s, 1H), 9.32 (dd, J = 6.8, 1.6 Hz, 1H), 8.63 (dd, J = 4.4, 1.6 Hz, 1H), 7.82- 7.76 (m, 2H), 7.73-7.71 (m, 1H), 7.62 (s, 2H), 7.34 (dd, J = 6.8, 4.0 Hz, 1H). | Not available |
| 5K | 2-[4- (Dimethylsulfamoyl)- 2- fluorophenyl]pyrazolo [1,5- a]pyrimidine-3- carboxylic acid | 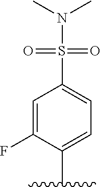 [SMILES: *c1ccc(S(=O)(=O)N(C)C)cc1F] | (400 MHz) δ 12.46 (s, 1H), 9.32 (dd, J = 6.8, 1.6 Hz, 1H), 8.87 (dd, J = 4.0, 1.6 Hz, 1H), 7.89- 7.85 (m, 1H), 7.74-7.70 (m, 1H), 7.37 (dd, J = 6.8, 4.0 Hz, 1H), 2.71 (s, 6H). | Not available |
| 5L | 2-(2-Fluoro-5- methylsulfonylphenyl) pyrazolo[1,5- a]pyrimidine-3- carboxylic acid | 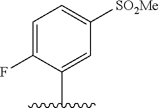 [SMILES: *c1cc(S(C)(=O)=O)ccc1F] | (400 MHz) δ 9.33 (dd, J = 7.2, 2.0 Hz, 1H), 8.87 (dd, J = 4.4, 2.0 Hz, 1H), 8.17-8.11 (m, 2H), 7.67-7.63 (m, 1H), 7.36 (dd, J = 7.2, 4.4 Hz, 1H), 3.32 (s, 3H). | Not available |

### 5M 2-(2-Fluoro-4-hydroxyphenyl)pyrazolo[1,5-a]pyrimidine-3-carboxylic Acid

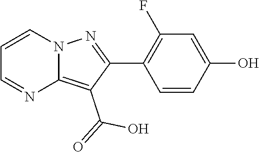 [SMILES: O=C(O)c1c(-c2ccc(O)cc2F)nn2cccnc12]

A solution of intermediate 4F (172 mg, 0.57 mmol) and LiOH (1 M aq., 1.71 mL, 1.71 mmol) in THF/MeOH (3:1; 2.82 mL) was heated at 50° C. overnight. Additional LiOH (1 M aq., 1.14 mL, 1.14 mmol) was added and the reaction heated at 60° C. overnight. Analogous workup and purification to intermediate 5A afforded a brown solid (155 mg, 99%). TLC R_{f} (200:8:1 CH₂Cl₂:EtOH:HCOOH)=0.47. ¹H NMR (400 MHz, DMSO-d₆) δ 12.23 (s, 1H), 10.19 (s, 1H), 9.25 (dd, J=7.0, 1.8 Hz, 1H), 8.79 (dd, J=4.2, 1.8 Hz, 1H), 7.38 (t, J=8.5 Hz, 1H), 7.27 (dd, J=7.0, 4.2 Hz, 1H), 6.74-6.62 (m, 2H).

### 6A 1-(4-Bromo-3-fluorophenyl)-2-methylpropan-2-ol

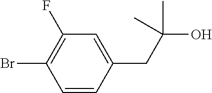

A solution of methyl 2-(4-bromo-3-fluorophenyl)acetate (1 g, 4.05 mmol) in THF (33 mL) was cooled to -78° C. and methylmagnesium bromide (3.4 M in 2-Me THF; 23.81 mL, 80.95 mmol) added dropwise over 10 min. The reaction was allowed to attain rt and stirred overnight. The reaction was quenched with sat. aq. NH₄Cl (50 mL), extracted with CH₂Cl₂ (2×50 mL), the combined organic extracts washed with brine (30 mL), dried (MgSO₄) and evaporated under reduced pressure. Purification by column chromatography (10-40% EtOAc in heptane) afforded a colourless oil (846 mg, 85%). ¹H NMR (400 MHz, DMSO-d₆) δ 7.56 (dd, J=8.1, 7.6 Hz, 1H), 7.20 (dd, J=10.3, 2.0 Hz, 1H), 7.00 (dd, J=8.2, 1.9 Hz, 1H), 4.41 (s, 1H), 2.64 (s, 2H), 1.05 (s, 6H). LRMS (APCI−) m/z 245.0, 247.0 [M-H]⁻.

### 7A 1-(4-Bromo-3-fluorophenyl)ethanol

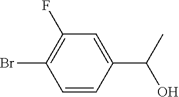

NaBH₄ (349 mg, 9.21 mmol) was added to a solution of 4-bromo-3-fluoroacetophenone (1 g, 4.61 mmol) in EtOH (20 mL) and the resulting suspension was left to stir at rt for 6 h. The reaction was quenched with water (40 mL), extracted with CH₂Cl₂ (2×50 mL), and the combined organic extracts washed with brine (20 mL), dried (MgSO₄) and evaporated under reduced pressure to afford a colourless oil which was used without further purification (955 mg, 95%). TLC R_{f} (1:1 EtOAc:Heptane)=0.68. ¹H NMR (400 MHz, DMSO-d₆) δ 7.67-7.57 (m, 1H), 7.31 (dd, J=10.2, 2.0 Hz, 1H), 7.14 (dd, J=8.3, 1.9 Hz, 1H), 5.38 (d, J=4.5 Hz, 1H), 4.78-4.66 (m, 1H), 1.30 (d, J=6.5 Hz, 3H).

### 8A 2-(4-Bromo-3-fluorophenyl)propan-2-ol

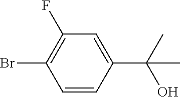

A solution of 4-bromo-3-fluoroacetophenone (1 g, 4.61 mmol) in THF was cooled to −78° C. and methylmagnesium bromide (3 M in Et₂O; 2 mL, 5.99 mmol) added dropwise. The reaction was allowed to attain rt and stirred for 1 h. The reaction was quenched with sat. aq. NH₄Cl (50 mL), extracted with CH₂Cl₂ (2×50 mL), and the combined organic extracts washed with brine (30 mL), dried (MgSO₄) and evaporated under reduced pressure. Purification by column chromatography (10-50% EtOAc in heptane) afforded a colourless oil (600 mg, 56%). TLC R_{f} (1:1 EtOAc:Heptane)=0.68. ¹H NMR (400 MHz, DMSO-d₆) δ 7.60 (dd, J=8.4, 7.4 Hz, 1H), 7.41 (dd, J=10.8, 2.1 Hz, 1H), 7.24 (dd, J=8.4, 2.1 Hz, 1H), 5.24 (s, 1H), 1.40 (s, 6H).

### 9A 2-(4-Bromo-3-fluorophenyl)ethanol

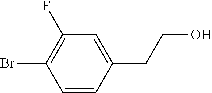 [SMILES: OCCc1ccc(Br)c(F)c1]

A solution of BH₃·DMSO (2 M in THF; 6.97 mL, 13.95 mmol) was added dropwise over 5 min to a solution of 4-bromo-3-fluorophenylacetic acid (2.50 g, 10.73 mmol) in THF (25 mL) and stirred at rt for 5.5 h. The reaction was quenched with MeOH (12 mL), the volatiles removed under reduced pressure and the crude residue purified by column chromatography (10-50% EtOAc in heptane) to afford a colourless oil (2.22 g, 94%). TLC R_{f} (1:1 EtOAc:heptane)=0.51. ¹H NMR (400 MHz, DMSO-d₆) δ 7.57 (dd, J=8.1, 7.6 Hz, 1H), 7.24 (dd, J=10.2, 2.0 Hz, 1H), 7.02 (ddd, J=8.2, 2.0, 0.6 Hz, 1H), 4.68 (t, J=5.2 Hz, 1H), 3.60 (td, J=6.7, 5.2 Hz, 2H), 2.71 (t, J=6.7 Hz, 2H).

### 10A 1-[3-Fluoro-4-(4,4,5,5-tetramethyl-1,3,2-dioxaborolan-2-yl)phenyl]2-methylpropan-2-ol

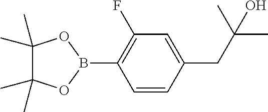 [SMILES: CC(C)(O)Cc1ccc(B2OC(C)(C)C(C)(C)O2)c(F)c1]

A reaction vessel was charged with intermediate 6A (750 mg, 3.04 mmol), bis(pinacolato)diboron (925 mg, 3.64 mmol), Pd(dppf)Cl₂ (222 mg, 0.30 mmol, 10 mol %) and KOAc (596 mg, 6.07 mmol) and sparged with N₂ (4×). 1,4-Dioxane (8 mL) which had been degassed with N₂ was added and the reaction heated at 80° C. for 21 h. The volatiles were removed under reduced pressure and the residue purified by column chromatography (10-50% EtOAc/heptane) to afford an off-white solid (500 mg, 56%). TLC R_{f} (2:1 EtOAc:heptane)=0.41. ¹H NMR (400 MHz, DMSO-d₆) δ 7.53 (dd, J=7.5, 6.6 Hz, 1H), 7.05 (dd, J=7.6, 1.3 Hz, 1H), 6.99 (dd, J=10.8, 1.3 Hz, 1H), 4.40 (s, 1H), 2.68 (s, 2H), 1.29 (s, 12H), 1.06 (s, 6H).

The following intermediate compounds were prepared in an analogous manner to intermediate 10A

TABLE 3
Preparatory Examples

| Preparatory | | | ¹H NMR & | LRMS m/z; |
|---|---|---|---|---|
| Example | Name | Structure | (400 MHz, DMSO-d₆) | TLC R_{f} |
| 10B | 1-[3-Fluoro-4- (4,4,5,5-tetramethyl- 1,3,2-dioxaborolan- 2-yl)phenyl]ethanol | 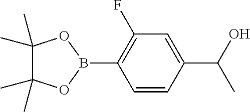 [SMILES: CC(O)c1ccc(B2OC(C)(C)C(C)(C)O2)c(F)c1] | 7.58 (dd, J = 7.6, 6.3 Hz, 1H), 7.20-7.12 (m, 1H), 7.09 (dt, J = 10.7, 0.9 Hz, 1H), 5.33 (d, J = 4.4 Hz, 1H), 4.73 (qd, J = 6.4, 4.4 Hz, 1H), 1.30 (d, J = 6.5 Hz, 3H), 1.28 (s, 12H). | 0.73 (1:1 EtOAc: heptane) |
| 10C | 2-[3-Fluoro-4- (4,4,5,5-tetramethyl- 1,3,2-dioxaborolan- 2-yl)phenyl]propan- 2-o1 | 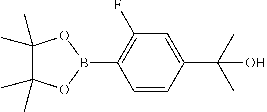 [SMILES: CC(C)(O)c1ccc(B2OC(C)(C)C(C)(C)O2)c(F)c1] | 7.57 (dd, J = 7.8, 6.5 Hz, 1H), 7.30-7.26 (m, 1H), 7.19 (dd, J = 11.3, 1.5 Hz, 1H), 5.18 (s, 1H), 1.40 (s, 6H), 1.28 (s, 12H). | 0.65 (1:1 EtOAc: heptane) |
| 10D | 2-[3-Fluoro-4- (4,4,5,5-tetramethyl- 1,3,2-dioxaborolan- 2-yl)phenyl]ethanol | 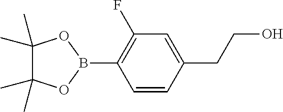 [SMILES: CC1(C)OB(c2ccc(CCO)cc2F)OC1(C)C] | 7.54 (dd, J = 7.5, 6.4 Hz, 1H), 7.09-6.96 (m, 2H), 4.71-4.64 (m, 1H), 3.65- 3.55 (m, 2H), 2.80-2.68 (m, 2H), 1.28 (s, 12H). | (APCI+) 267.2 [M + H]⁺ |

The following intermediate compounds were prepared in an analogous manner to intermediate 4A from the corresponding boronic acid pinacol ester. Intermediates 11A-D were prepared with heating by MWI at 80° C. for 30 min .

TABLE 4
Preparatory examples prepared via general Suzuki-Miyaura procedure.

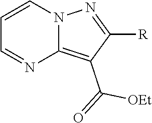

| | | | | LRMS |
|---|---|---|---|---|
| Preparatory | | | ¹H NMR δ | m/z |
| Example | Name | R | (400 MHz, DMSO-d₆) | (APCI+) |
| 11A | Ethyl 2-[2-fluoro-4- (2-hydroxy-2- methylpropyl)phenyl] pyrazolo[1,5- a]pyrimidine-3- carboxylate | 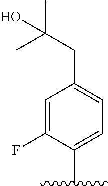 [SMILES: *c1ccc(CC(C)(C)O)cc1F] | 9.31 (dd, J = 7.0, 1.7 Hz, 1H), 8.85 (dd, J = 4.2, 1.7 Hz, 1H), 7.48 (t, J = 7.8 Hz, 1H), 7.33 (dd, J = 7.0, 4.2 Hz, 1H), 7.18 (dd, J = 4.4, 1.4 Hz, 1H), 7.16 (s, 1H), 4.48 (s, 1H), 4.17 (q, J = 7.1 Hz, 2H), 2.75 (s, 2H), 1.30- 0.88 (m, 9H). | 358.1 [M + H]⁺ |
| 11B | Ethyl 2-[2-fluoro-4- (1- hydroxyethyl)phenyl] pyrazolo[1,5- a]pyrimidine-3- carboxylate | 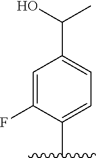 [SMILES: *c1ccc(C(C)O)cc1F] | 9.31 (dd, J = 7.0, 1.7 Hz, 1H), 8.85 (dd, J = 4.2, 1.7 Hz, 1H), 7.53 (t, J = 7.6 Hz, 1H), 7.35-7.23 (m, 3H), 5.40 (d, J = 4.4 Hz, 1H), 4.88-4.76 (m, 1H), 4.17 (q, J = 7.1 Hz, 2H), 1.37 (d, J = 6.4 Hz, 3H), 1.13 (t, J = 7.1 Hz, 3H) | 330.3 [M + H]⁺ |
| 11C | Ethyl 2-[2-fluoro-4- (2-hydroxypropan- 2- yl)phenyl]pyrazolo [1,5-a]pyrimidine-3- carboxylate | 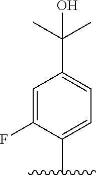 [SMILES: *c1ccc(C(C)(C)O)cc1F] | 9.31 (dd, J = 7.0, 1.8 Hz, 1H), 8.86 (dd, J = 4.2, 1.8 Hz, 1H), 7.53 (t, J = 7.8 Hz, 1H), 7.43-7.35 (m, 2H), 7.33 (dd, J = 7.0, 4.2 Hz, 1H), 5.27 (s, 1H), 4.18 (q, J = 7.1 Hz, 2H), 1.48 (s, 6H), 1.14 (t, J = 7.1 Hz, 3H). | 344.3 [M + H]⁺ |
| 11D | Ethyl 2-[2-fluoro-4- (2- hydroxyethyl)phenyl] pyrazolo[1,5- a]pyrimidine-3- carboxylate | 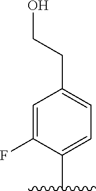 [SMILES: *c1ccc(CCO)cc1F] | 9.30 (dd, J = 7.0, 1.7 Hz, 1H), 8.85 (dd, J = 4.2, 1.8 Hz, 1H), 7.49 (t, J = 7.8 Hz, 1H), 7.32 (dd, J = 7.0, 4.2 Hz, 1H), 7.24-7.14 (m, 2H), 4.75 (t, J = 5.1 Hz, 1H), 4.18 (q, J = 7.1 Hz, 2H), 3.68 (td, J = 6.8, 5.1 Hz, 2H), 2.82 (t, J = 6.8 Hz, 2H), 1.14 (t, J = 7.1 Hz, 3H). | 330.4 [M + H]⁺ |

### 12A 2-[2-Fluoro-4-(2-hydroxy-2-methylpropyl)phenyl]pyrazolo[1,5-a]pyrimidine-3-carboxylic acid

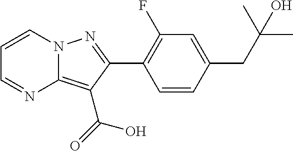 [SMILES: CC(C)(O)Cc1ccc(-c2nn3cccnc3c2C(=O)O)c(F)c1]

A solution of intermediate 11A (325 mg, 0.91 mmol) and LiOH (1 M aq., 2.73 mL, 2.73 mmol) in THF/MeOH (3:1; 5.3 mL) was heated at 50° C. overnight. The solvents were removed under reduced pressure and the residue diluted with water (5 mL) and acidified with 1 M aq. HCl (3.64 mL). The resultant precipitate was filtered, washed with water and dried under reduced pressure to afford an orange solid (230 mg, 77%) which was used without further purification. ¹H NMR (400 MHz, DMSO-d₆) δ 12.25 (s, 1H), 9.27 (dd, J=7.0, 1.8 Hz, 1H), 8.81 (dd, J=4.2, 1.8 Hz, 1H), 7.46 (t, J=7.9 Hz, 1H), 7.29 (dd, J=7.0, 4.2 Hz, 1H), 7.19-7.11 (m, 2H), 4.46 (s, 1H), 2.74 (s, 2H), 1.08 (s, 6H). LRMS (APCI+) m/z 330.2 [M+H]⁺.

### 12B 2-[2-Fluoro-4-(2-hydroxypropan-2-yl)phenyl]pyrazolo[1,5-a]pyrimidine-3-carboxylic acid

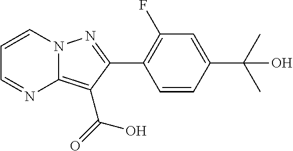 [SMILES: CC(C)(O)c1ccc(-c2nn3cccnc3c2C(=O)O)c(F)c1]

A solution of intermediate 11C (358 mg, 1.04 mmol) and LiOH (1 M aq., 3.13 mL, 3.13 mmol) in THF/MeOH (3:1; 6.5 mL) was heated at 50° C. for 25 h, then stirred at rt over the weekend. The reaction was quenched with 1 M aq. HCl (4.17 mL), resulting in the formation of a precipitate, and diluted with water (20 mL). The suspension was stirred for 5 minutes, filtered, washed with water (−10 mL), and dried under reduced pressure to afford an off-white solid (194 mg). The filtrate was extracted with EtOAc (2×25 mL), the combined organics washed with brine (20 mL), dried (MgSO₄) and the solvent removed under reduced pressure to afford a 92 mg of an off-white solid. The solids were combined and used without further purification to give an off-white solid (286 mg, 87%). ¹H NMR (400 MHz, DMSO-d₆) δ 12.30 (s, 1H), 9.28 (dd, J=7.0, 1.8 Hz, 1H), 8.82 (dd, J=4.2, 1.8 Hz, 1H), 7.51 (t, J=7.9 Hz, 1H), 7.41-7.33 (m, 2H), 7.29 (dd, J=7.0, 4.2 Hz, 1H), 5.26 (s, 1H), 1.47 (s, 6H). LRMS (APCI+) m/z 316.1 [M+H]+.

### 12C 2-[2-Fluoro-4-(2-hydroxyethyl)phenyl]pyrazolo[1,5-a]pyrimidine-3-carboxylic acid

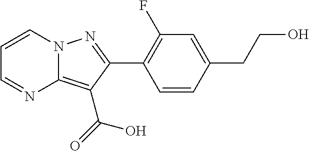 [SMILES: O=C(O)c1c(-c2ccc(CCO)cc2F)nn2cccnc12]

A solution of intermediate 11D (254 mg, 0.77 mmol) and LiOH (1 M aq., 2.31 mL, 2.31 mmol) in THF/MeOH (4:1; 5 mL) was heated at 50° C. overnight. The reaction was quenched with 1 M aq. HCl (3.09 mL, 3.09 mmol) and diluted with water (20 mL). The mixture was extracted with EtOAc (2×50 mL), the combined organic extracts washed with brine (30 mL), dried (MgSO₄) and the solvent removed under reduced pressure to afford an orange solid (110 mg). The aqueous layers were subsequently extracted with CHCl_{3/}iPrOH (3:1; 2×30 mL), and organic extracts were washed with brine (30 mL), dried (MgSO₄) and the solvent removed under reduced pressure to afford an orange solid (42 mg). The solids were combined and used without further purification. Orange solid (152 mg, 66%). TLC R_{f} (EtOAc)=0.08. ¹H NMR (400 MHz, DMSO-d₆) 12.30 (s, 1H), 9.28 (dd, J=7.0, 1.8 Hz, 1H), 8.81 (dd, J=4.2, 1.8 Hz, 1H), 7.47 (t, J=7.8 Hz, 1H), 7.29 (dd, J=7.0, 4.2 Hz, 1H), 7.22-7.13 (m, 2H), 4.75 (t, J=5.1 Hz, 1H), 3.68 (td, J=6.7, 5.0 Hz, 2H), 2.81 (t, J=6.8 Hz, 2H).

### 12D 2-[2-Fluoro-4-(1-hydroxyethyl)phenyl]pyrazolo[1,5-a]pyrimidine-3-carboxylic acid

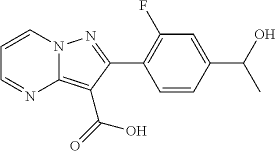 [SMILES: CC(O)c1ccc(-c2nn3cccnc3c2C(=O)O)c(F)c1]

A solution of intermediate 11B (441 mg, 1.34 mmol) and LiOH (1 M aq., 4.02 mL, 4.02 mmol) in THF/MeOH (4:1; 8 mL) was heated at 50° C. overnight. The reaction was quenched with 1 M aq. HCl (5.36 mL) and diluted with water (50 mL). The mixture was extracted with EtOAc (2×50 mL), the combined organic extracts washed with brine (50 mL), dried (MgSO₄) and the solvent removed under reduced pressure to afford an orange solid which was used without further purification (366 mg, 91%). ¹H NMR (400 MHz, DMSO-d₆) δ 12.30 (s, 1H), 9.28 (dd, J=7.0, 1.8 Hz, 1H), 8.82 (dd, J=4.2, 1.7 Hz, 1H), 7.52 (t, J=7.8 Hz, 1H), 7.38-7.20 (m, 3H), 5.38 (s, 1H), 4.80 (q, J=6.4 Hz, 1H), 1.38 (d, J=6.5 Hz, 3H). LRMS (APCI+) m/z 301.9 [M+H]⁺.

### 13A [3-Fluoro-4-[3-[[(3S)-9-fluoro-2-oxo-5-phenyl-1,3-dihydro-1,4-benzodiazepin-3-yl]carbamoyl]pyrazolo[1,5-a]pyrimidin-2-yl]phenyl]methyl 2-[(2-methylpropan-2-yl)oxycarbonylamino]acetate

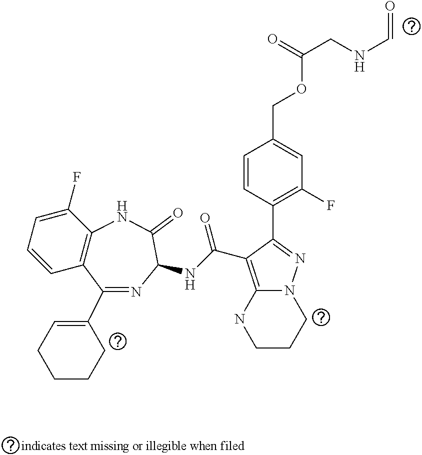 [SMILES: O=CNCC(=O)OCc1ccc(-c2nn3c(c2C(=O)N[C@H]2N=C(C4=CCCCC4)c4cccc(F)c4NC2=O)NCCC3)c(F)c1]

N-Ethyl-3-(3-dimethylaminopropyl)carbodiimide hydrochloride (96 mg, 0.5 mmol) and 4-dimethylaminopyridine (6.8 mg, 0.06 mmol) were added to a solution of 2-[2-fluoro-4-(hydroxymethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide (150 mg, 0.28 mmol) and Boc-Gly-OH (73 mg, 0.42 mmol) in DMF (1.5 mL) and stirred at 30° C. for 20 h. The reaction was quenched by the addition of water (10 mL), the resulting precipitate collected by filtration, washed with water, and dried at the pump. Purification by flash chromatography [5-25% (EtOH:CH₂Cl₂:NRIOH; 50:8:1) in CH₂Cl₂] afforded a white solid (182 mg, 94%). ¹H NMR (400 MHz, DMSO-d₆) δ 10.98 (s, 1H), 9.59 (d, J=7.9 Hz, 1H), 9.43 (dd, J=7.0, 1.7 Hz, 1H), 9.00 (dd, J=4.3, 1.7 Hz, 1H), 7.63-7.56 (m, 1H), 7.55-7.38 (m, 7H), 7.35-7.21 (m, 4H), 7.18-7.11 (m, 1H), 5.52 (d, J=7.9 Hz, 1H), 5.19 (s, 2H), 3.77 (d, J=6.2 Hz, 2H), 1.36 (s, 9H). LRMS (APCI+) m/z 696.3 [M+H]⁺.

The following intermediate compounds were prepared in an analogous manner to intermediate 13A from 2-[2-fluoro-4-(hydroxymethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide or 2-[2-fluoro-4-(hydroxymethyl)phenyl]-N-[(3S)-2-oxo-5-phenyl-1,3-dihydro-1,4-benzodiazepin-3-yl]pyrazolo[1,5-a]pyrimidine-3-carboxamide.

TABLE 5
Preparatory Examples

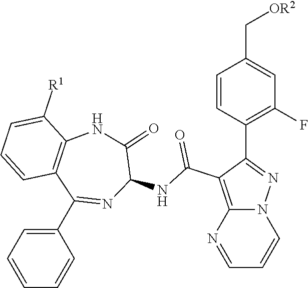

| | | | | | LRMS |
|---|---|---|---|---|---|
| Preparatory | | | | ¹H NMR δ | m/z |
| Example | Name | R¹ | R² | (400 MHz, DMSO-d₆) | (APCI+) |
| 13B | [3-Fluoro-4-[3-[[(3S)- 2-oxo-5-phenyl-1,3- dihydro-1,4- benzodiazepin-3- | H | 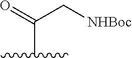 [SMILES: *C(=O)CNC(=O)OC(C)(C)C] | 11.02 (s, 1H), 9.58 (d, J = 8.0 Hz, 1H), 9.43 (dd, J = 7.0, 1.7 Hz, 1H), 9.00 (dd, J = 4.3, 1.7 Hz, 1H), 7.68- 7.60 (m, 1H), 7.55-7.39 (m, | 678.5 [M + H]⁺ |
| | yl]carbamoyl]pyrazolo | | | 7H), 7.36-7.21 (m, 6H), 5.44 (d, | |
| | [1,5-a]pyrimidin-2- | | | J = 8.0 Hz, 1H), 5.19 (s, 2H), 3.77 | |
| | yl]phenyl]methyl 2- | | | (d, J = 6.2 Hz, 2H), 1.36 (s, 9H) | |
| | [(2-methylpropan-2- | | | | |
| | yl)oxycarbonylamino] | | | | |
| | acetate | | | | |
| 13C | [3-Fluoro-4-[3-[[(3S)- 9-fluoro-2-oxo-5- phenyl-1,3-dihydro- 1,4-benzodiazepin-3- yl]carbamoyl]pyrazolo [1,5-a]pyrimidin-2- | F | 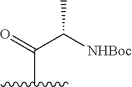 [SMILES: *C(=O)[C@H](C)NC(=O)OC(C)(C)C] | 10.98 (s, 1H), 9.59 (d, J = 8.0 Hz, 1H), 9.43 (dd, J = 7.0, 1.7 Hz, 1H), 9.00 (dd, J = 4.3, 1.7 Hz, 1H), 7.64- 7.56 (m, 1H), 7.56-7.36 (m, 8H), 7.31 (td, J = 8.1, 5.0 Hz, 1H), 7.28-7.20 (m, 2H), 7.17-7.10 | 710.2 [M + H]⁺ |
| | yl]phenyl]methyl (2S)- | | | (m, 1H), 5.53 (d, J = 8.0 Hz, 1H), | |
| | 2-[(2-methylpropan-2- | | | 5.23-5.11 (m, 2H), 4.14-4.05 | |
| | yl)oxycarbonylamino] | | | (m, 1H), 1.35 (s, 9H), 1.27 (d, J = | |
| | propanoate | | | 7.3 Hz, 3H). | |
| 13D | [3-Fluoro-4-[3-[[(3S)- 9-fluoro-2-oxo-5- phenyl-1,3-dihydro- 1,4-benzodiazepin-3- yl]carbamoyl]pyrazolo [1,5-a]pyrimidin-2- | F | 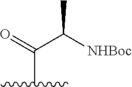 [SMILES: *C(=O)[C@@H](C)NC(=O)OC(C)(C)C] | 10.98 (s, 1H), 9.59 (d, J = 8.0 Hz, 1H), 9.43 (dd, J = 7.0, 1.7 Hz, 1H), 9.00 (dd, J = 4.3, 1.7 Hz, 1H), 7.64- 7.54 (m, 1H), 7.57-7.35 (m, 8H), 7.31 (td, J = 8.1, 5.1 Hz, 1H), 7.29-7.20 (m, 2H), 7.18-7.10 | 710.3 [M + H]⁺ |
| | yl]phenyl]methyl (2R)- | | | (m, 1H), 5.53 (d, J = 7.9 Hz, 1H), | |
| | 2-[(2-methylpropan-2- | | | 5.25-5.10 (m, 2H), 4.09 (p, J = | |
| | yl)oxycarbonylamino] | | | 7.3 Hz, 1H), 1.35 (s, 9H), 1.27 (d, | |
| | propanoate | | | J = 7.4 Hz, 3H). | |
| 13E | [3-Fluoro-4-[3-[[(3S)- 9-fluoro-2-oxo-5- phenyl-1,3-dihydro- 1,4-benzodiazepin-3- yl]carbamoyl]pyrazolo [1,5-a]pyrimidin-2- yl]phenyl]methyl (2S)- | F | 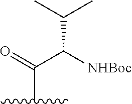 [SMILES: *C(=O)[C@@H](NC(=O)OC(C)(C)C)C(C)C] | 10.98 (s, 1H), 9.59 (d, J = 8.0 Hz, 1H), 9.43 (dd, J = 7.0, 1.7 Hz, 1H), 9.00 (dd, J = 4.3, 1.7 Hz, 1H), 7.59 (ddd, J = 10.4, 8.2, 1.3 Hz, 1H), 7.57-7.38 (m, 7H), 7.36-7.21 (m, 4H), 7.20-7.11 (m, 1H), 5.53 (d, J = 7.9 Hz, 1H), 5.25-5.12 (m, | 738.2 [M + H]⁺ |
| | 3-methyl-2-[(2- | | | 2H), 3.89 (dd, J = 8.0, 6.7 Hz, 1H), | |
| | methylpropan-2- | | | 2.10-1.99 (m, 1H), 1.35 (s, 9H), | |
| | yl)oxycarbonylamino] | | | 0.87 (dd, J = 6.8, 1.5 Hz, 6H) | |
| | butanoate | | | | |
| 13F | [3-Fluoro-4-[3-[[(3S)- 9-fluoro-2-oxo-5- phenyl-1,3-dihydro- 1,4-benzodiazepin-3- yl]carbamoyl]pyrazolo [1,5-a]pyrimidin-2- yl]phenyl]methyl (2R)- | F | 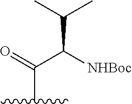 [SMILES: *C(=O)[C@H](NC(=O)OC(C)(C)C)C(C)C] | 10.98 (s, 1H), 9.59 (d, J = 8.0 Hz, 1H), 9.43 (dd, J = 7.0, 1.7 Hz, 1H), 9.00 (dd, J = 4.3, 1.7 Hz, 1H), 7.64- 7.54 (m, 1H), 7.57-7.38 (m, 7H), 7.36-7.21 (m, 4H), 7.15 (dt, J = 7.9, 1.1 Hz, 1H), 5.52 (d, J = 8.0 Hz, 1H), 5.26-5.12 (m, 2H), | 738.3 [M + H]⁺ |
| | 3-methyl-2-[(2- | | | 3.89 (dd, J = 8.0, 6.6 Hz, 1H), 2.11- | |
| | methylpropan-2- | | | 1.99 (m, 1H), 1.35 (s, 9H), 0.87 | |
| | yl)oxycarbonylamino] | | | (dd, J = 6.7, 1.5 Hz, 6H). | |
| | butanoate | | | | |
| 13G | [3-Fluoro-4-[3-[[(3S)- 9-fluoro-2-oxo-5- phenyl-1,3-dihydro- 1,4-benzodiazepin-3- yl]carbamoyl]pyrazolo [1,5-a]pyrimidin-2- yl]phenyl]methyl (2S)- 4-methyl-2-[(2- methylpropan-2- | F | 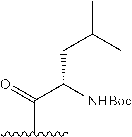 [SMILES: *C(=O)[C@H](CC(C)C)NC(=O)OC(C)(C)C] | 10.98 (s, 1H), 9.59 (d, J = 8.0 Hz, 1H), 9.43 (dd, J = 7.0, 1.7 Hz, 1H), 9.00 (dd, J = 4.3, 1.7 Hz, 1H), 7.64- 7.54 (m, 1H), 7.57-7.38 (m, 7H), 7.38-7.26 (m, 2H), 7.29- 7.18 (m, 2H), 7.18-7.09 (m, 1H), 5.53 (d, J = 7.9 Hz, 1H), 5.18 (d, J = 2.1 Hz, 2H), 4.11-3.99 (m, 1H), 1.71-1.51 (m, 2H), 1.52-1.39 | 752.2 [M + H]⁺ |
| | yl)oxycarbonylamino] | | | (m, 1H), 1.34 (s, 9H), 0.85 (dd, J = | |
| | pentanoate | | | 12.5, 6.4 Hz, 6H). | |
| 13H | [3-Fluoro-4-[3-[[(3S)- 9-fluoro-2-oxo-5- phenyl-1,3-dihydro- 1,4-benzodiazepin-3- yl]carbamoyl]pyrazolo [1,5-a]pyrimidin-2- yl]phenyl methyl (2R)- 4-methyl-2-[(2- methylpropan-2- | F | 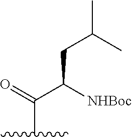 [SMILES: *C(=O)[C@@H](CC(C)C)NC(=O)OC(C)(C)C] | 10.98 (s, 1H), 9.59 (d, J = 7.9 Hz, 1H), 9.43 (dd, J = 7.0, 1.7 Hz, 1H), 9.00 (dd, J = 4.3, 1.7 Hz, 1H), 7.63- 7.56 (m, 1H), 7.56-7.39 (m, 7H), 7.37-7.28 (m, 2H), 7.27- 7.20 (m, 2H), 7.14 (dd, J = 7.9, 1.3 Hz, 1H), 5.53 (d, J = 7.9 Hz, 1H), 5.18 (d, J = 2.2 Hz, 2H), 4.12- 3.99 (m, 1H), 1.70-1.52 (m, 2H), | 752.3 [M + H]⁺ |
| | yl)oxycarbonylamino] | | | 1.51-1.40 (m, 1H), 1.34 (s, 9H), | |
| | pentanoate | | | 0.85 (dd, J = 12.5, 6.4 Hz, 6H). | |

### 14A [3-Fluoro-4-[3-[[(3S)-2-oxo-5-phenyl-1,3-dihydro-1,4-benzodiazepin-3-yl]carbamoyl]pyrazolo[1,5-a]pyrimidin-2-yl]phenyl]methyl (2S)-3-methyl-2-[(2-methylpropan-2-yl)oxycarbonylamino]butanoate

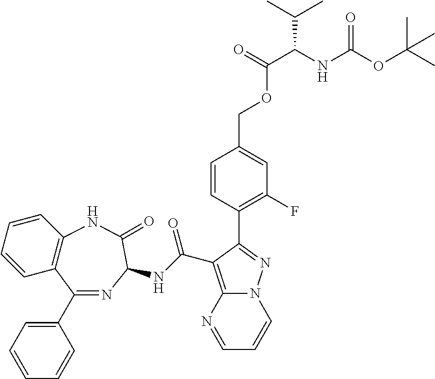 [SMILES: CC(C)[C@H](NC(=O)OC(C)(C)C)C(=O)OCc1ccc(-c2nn3cccnc3c2C(=O)N[C@H]2N=C(c3ccccc3)c3ccccc3NC2=O)c(F)c1]

Carbonyldiimidazole (47 mg, 0.29 mmol) and DIPEA (100 μL, 0.58 mmol) were added successively to a cooled (0° C.) solution of Boc-Val-OH (63 mg, 0.29 mmol) in MeCN (1 mL) and the resulting mixture allowed to warm up to rt and stirred for 1 h. 2-[2-Fluoro-4-(hydroxymethyl)phenyl]-N-[(3S)-2-oxo-5-phenyl-1,3-dihydro-1,4-benzodiazepin-3-yl]pyrazolo[1,5-a]pyrimidine-3-carboxamide (100 mg, 0.19 mmol) was added and resulting mixture stirred at rt for 20 h. The reaction was quenched with water (20 mL), extracted with EtOAc (2×30 mL), the combined organic extracts washed with brine (30 mL), dried (MgSO₄) and evaporated under reduced pressure. Purification by flash chromatography (50-100% EtOAc in Heptane) afforded a white solid (70 mg, 51%). ¹H NMR (400 MHz, DMSO-d₆) δ 11.01 (s, 1H), 9.58 (d, J=8.1 Hz, 1H), 9.43 (dd, J=7.0, 1.7 Hz, 1H), 9.00 (dd, J=4.3, 1.7 Hz, 1H), 7.68-7.62 (m, 1H), 7.54-7.48 (m, 2H), 7.48-7.39 (m, 5H), 7.36-7.21 (m, 6H), 5.44 (d, J=8.0 Hz, 1H), 5.28-5.09 (m, 2H), 3.89 (dd, J=8.0, 6.7 Hz, 1H), 2.03 (h, J=6.9 Hz, 1H), 1.35 (s, 9H), 0.87 (dd, J=6.8, 1.4 Hz, 6H). LRMS (APCI+) m/z 720.5 [M+H]⁺.

### EXAMPLES

### 1. 2-[2-Fluoro-4-(hydroxymethyl)phenyl]-N-[(35)-9-fluoro-2-oxo-5-phenyl-1,3-dihydro-1,4-benzodiazepin-3-yl]pyrazolo[1,5-a]pyrimidine-3-carboxamide

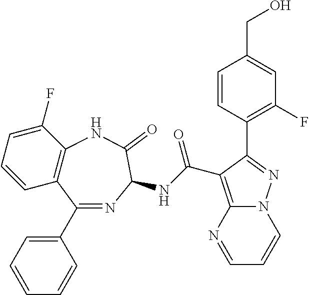 [SMILES: O=C(N[C@H]1N=C(c2ccccc2)c2cccc(F)c2NC1=O)c1c(-c2ccc(CO)cc2F)nn2cccnc12]

HATU (85 mg, 0.22 mmol) was added to a suspension of intermediate 5A (55 mg, 0.19 mmol) and DIPEA (65 μL, 0.37 mmol) in DMF (1.5 mL) and stirred at rt for 5 min. (3S)-3-amino-9-fluoro-5-phenyl-1,3-dihydro-1,4-benzodiazepin-2-one (50 mg, 0.19 mmol) was added and the reaction stirred overnight at rt. The reaction was quenched with water, and the resultant precipitate filtered, washing with water. The precipitate was dissolved in EtOAc, washed with 0.1 M HCl (2×10 mL) and brine (10 mL), dried MgSO₄, and the solvent removed under reduced pressure. Purification by flash chromatography [13-37% (EtOH:CH₂Cl₂:NH₄OH; 50:8:1) in CH₂Cl₂] afforded a white solid (72 mg, 72%). ¹H NMR (600 MHz, DMSO-d₆) δ 10.95 (s, 1H), 9.59 (d, J=7.9 Hz, 1H), 9.42 (dd, J=7.0, 1.6 Hz, 1H), 8.99 (dd, J=4.3, 1.7 Hz, 1H), 7.61-7.56 (m, 1H), 7.55-7.49 (m, 3H), 7.48-7.43 (m, 3H), 7.41 (dd, J=7.0, 4.3 Hz, 1H), 7.33-7.28 (m, 1H), 7.21-7.18 (m, 1H), 7.17-7.13 (m, 2H), 5.52 (d, J=7.9 Hz, 1H), 5.37 (t, J=5.8 Hz, 1H), 4.56 (d, J=5.7 Hz, 2H). LRMS (APCI+) m/z 539.2 [M+H]⁺.

The following compounds of the invention were prepared with (3S)-3-amino-9-fluoro-5-phenyl-1,3-dihydro-1,4-benzodiazepin-2-one or (3S)-3-amino-5-phenyl-1,3-dihydro-1,4-benzodiazepin-2-one by the amide coupling procedure described for the compound of Example 1. Example 3 was subject to additional repeat purification by flash chromatography [15-22% (EtOH:CH₂Cl₂:NH₄OH; 50:8:1) in CH₂Cl₂] followed by preparative HPLC (Method 1: MeCN/water with 0.2% v/v formic acid, 25-75% for 16 min).

TABLE 5
Examples compounds prepared by amide coupling procedure A

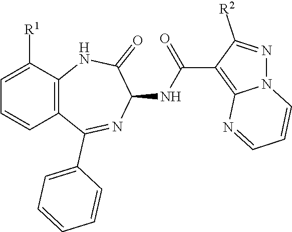

| | | | | | LRMS |
|---|---|---|---|---|---|
| | | | | ¹H NMR δ | m/z |
| Example | Name | R¹ | R² | (DMSO-d₆) | (APCI+) |
| 2 | 2-[2-Fluoro-4- (hydroxymethyl) phenyl]-N-[(3S)- 2-oxo- 5-phenyl-1,3- dihydro-1,4- benzodiazepin-3- yl]pyrazolo[1,5- a]pyrimidine-3- carboxamide | H | 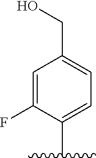 [SMILES: *c1ccc(CO)cc1F] | (400 MHz) δ 11.01 (s, 1H), 9.59 (d, J = 8.0 Hz, 1H), 9.42 (dd, J = 7.0, 1.7 Hz, 1H), 8.99 (dd, J = 4.3, 1.7 Hz, 1H), 7.64 (ddd, J = 8.6, 7.1, 1.7 Hz, 1H), 7.54-7.38 (m, 7H), 7.34-7.22 (m, 3H), 7.22-7.12 (m, 2H), 5.44 (d, J = 8.0 Hz, 1H), 5.39 (t, J = 5.8 Hz, 1H), 4.55 (d, J = 5.8 Hz, 2H). | 563.0 [M + H]⁺ |
| 3 | N-[(3S)-9-fluoro-2- oxo-5-phenyl-1,3- dihydro-1,4- benzodiazepin-3- yl]-2-[1-(2,2,2- trifluoroethyl)pyraz ol-4- yl]pyrazolo[1,5- a]pyrimidine-3- carboxamide | F | 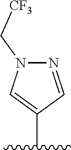 [SMILES: *c1cnn(CC(F)(F)F)c1] | (600 MHz) δ 10.98 (s, 1H), 10.01 (d, J = 7.7 Hz, 1H), 9.36 (dd, J = 7.0, 1.7 Hz, 1H), 9.01 (s, 1H), 8.92 (dd, J = 4.3, 1.7 Hz, 1H), 8.29 (s, 1H), 7.65- 7.60 (m, 2H), 7.57-7.52 (m, 3H), 7.48-7.44 (m, 2H), 7.38- 7.32 (m, 2H), 7.22 (d, J = 7.9 Hz, 1H), 5.67 (d, J = 7.7 Hz, 1H), 5.28-5.21 (m, 2H). | 563.0 [M + H]⁺ |
| 4 | N-[(3S)-9-fluoro-2- oxo-5-phenyl-1,3- dihydro-1,4- benzodiazepin-3- yl]-2-(1-propan-2- ylpyrazol-4- yl)pyrazolo[1,5- a]pyrimidine-3- carboxamide | F | 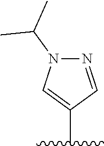 [SMILES: *c1cnn(C(C)C)c1] | (400 MHz) δ 11.01 (s, 1H), 10.02 (d, J = 7.8 Hz, 1H), 9.34 (dd, J = 6.9, 1.7 Hz, 1H), 8.90 (dd, J = 4.3, 1.6 Hz, 1H), 8.76 (d, J = 0.6 Hz, 1H), 8.19 (d, J = 0.6 Hz, 1H), 7.64 (ddd, J = 10.4, 8.2, 1.4 Hz, 1H), 7.53 (dt, J = 7.3, 1.2 Hz, 3H), 7.49-7.43 (m, 2H), 7.35 (ddd, J = 8.1, 7.5, | 522.8 [M + H]⁺ |
| | | | | 4.7 Hz, 2H), 7.24-7.19 (m, 1H), | |
| | | | | 5.67 (d, J = 7.8 Hz, 1H), 4.58 (p, | |
| | | | | J = 6.6 Hz, 1H), 1.42 (d, J = | |
| | | | | 6.7 Hz, 6H). | |
| 5 | N-[(3S)-2-oxo-5- phenyl-1,3-dihydro- 1,4-benzodiazepin- 3-yl]-2-(1-propan-2- ylpyrazol-4- yl)pyrazolo[1,5- a]pyrimidine-3- carboxamide | H | 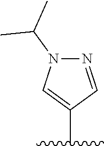 [SMILES: *c1cnn(C(C)C)c1] | (400 MHz) δ 11.04 (s, 1H), 10.00 (d, J = 8.0 Hz, 1H), 9.34 (dd, J = 6.9, 1.6 Hz, 1H), 8.89 (dd, J = 4.3, 1.7 Hz, 1H), 8.77 (d, J = 0.6 Hz, 1H), 8.19 (d, J = 0.6 Hz, 1H), 7.68 (ddd, J = 8.1, 7.2, 1.7 Hz, 1H), 7.55-7.42 (m, 5H), 7.40-7.27 (m, 4H), 5.60 (d, J = 8.0 Hz, 1H), 4.57 (p, J = | 505.0 [M + H]⁺ |
| | | | | 6.7 Hz, 1H), 1.42 (d, J = 6.7, | |
| | | | | 6H). | |
| 6 | N-[(3S)-9-fluoro-2- oxo-5-phenyl-1,3- dihydro-1,4- benzodiazepin-3- yl]-2-[1-(oxan-4- yl)pyrazol-4- yl]pyrazolo[1,5- a]pyrimidine-3- carboxamide | F | 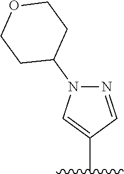 [SMILES: *c1cnn(C2CCOCC2)c1] | (600 MHz) δ 10.98 (s, 1H), 10.00 (d, J = 7.8 Hz, 1H), 9.33 (dd, J = 6.9, 1.7 Hz, 1H), 8.90 (dd, J = 4.3, 1.7 Hz, 1H), 8.81 (d, J = 0.6 Hz, 1H), 8.21 (d, J = 0.6 Hz, 1H), 7.65-7.61 (m, 1H), 7.56-7.52 (m, 3H), 7.48- 7.44 (m, 2H), 7.37-7.33 (m, 2H), 7.22 (d, J = 7.6 Hz, 1H), 5.67 (d, J = 7.8 Hz, 1H), 4.52- 4.46 (m, 1H), 3.97-3.93 (m, | 565.0 [M + H]⁺ |
| | | | | 2H), 3.47-3.41 (m, 2H), 2.00- | |
| | | | | 1.91 (m, 4H). | |
| 7 | 2-(2-Fluoro-4- methylsulfonylphenyl)- N-[(3S)-9- fluoro-2-oxo-5- phenyl-1,3-dihydro- 1,4-benzodiazepin- 3-yl]pyrazolo[1,5- a]pyrimidine-3- carboxamide | F | 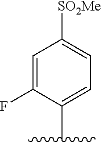 [SMILES: *c1ccc(S(C)(=O)=O)cc1F] | (400 MHz) δ 10.99 (s, 1H), 9.58 (d, J = 7.9 Hz, 1H), 9.47 (dd, J = 7.0, 1.7 Hz, 1H), 9.04 (dd, J = 4.3, 1.6 Hz, 1H), 7.89-7.78 (m, 3H), 7.64-7.40 (m, 7H), 7.36- 7.27 (m, 1H), 7.17-7.12 (m, 1H), 5.52 (d, J = 7.9 Hz, 1H), 3.33 (s, 3H). | 586.6 [M + H]⁺ |
| 8 | 2-(2-Fluoro-4- methylsulfonylphenyl)- N-[(3S)-2-oxo- 5-phenyl-1,3- dihydro-1,4- benzodiazepin-3- yl]pyrazolo[1,5- a]pyrimidine-3- carboxamide | H | 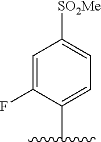 [SMILES: *c1ccc(S(C)(=O)=O)cc1F] | (400 MHz) δ 11.03 (s, 1H), 9.57 (d, J = 8.0 Hz, 1H), 9.47 (dd, J = 7.0, 1.7 Hz, 1H), 9.04 (dd, J = 4.3, 1.7 Hz, 1H), 7.88-7.80 (m, 3H), 7.67-7.61 (m, 1H), 7.54- 7.41 (m, 6H), 7.34-7.23 (m, 3H), 5.44 (d, J = 8.0 Hz, 1H), 3.33 (s, 3H). | 569.1 [M + H]⁺ |
| 9 | 2-[2-Fluoro-5- (hydroxymethyl) phenyl]-N-[(3S)- 2-oxo- 5-phenyl-1,3- dihydro-1,4- benzodiazepin-3- yl]pyrazolo[1,5- a]pyrimidine-3- | H | 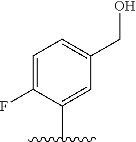 [SMILES: *c1cc(CO)ccc1F] | (400 MHz) δ 11.01 (s, 1H), 9.58 (d, J = 8.1 Hz, 1H), 9.42 (dd, J = 7.0, 1.7 Hz, 1H), 8.99 (dd, J = 4.3, 1.7 Hz, 1H), 7.64 (ddd, J = 8.6, 7.1, 1.7 Hz, 1H), 7.56-7.36 (m, 8H), 7.35-7.21 (m, 3H), 7.17 (dd, J = 9.8, 8.4 Hz, 1H), 5.45 (d, J = 8.1 Hz, 1H), 5.29 (t, J = 5.8 Hz, 1H), 4.51 (d, J = | 521.1 [M + H]⁺ |
| | carboxamide | | | 5.7 Hz, 2H). | |
| 10 | 2-[2-Fluoro-5- (hydroxymethyl) phenyl]-N-[(3S)-9- fluoro-2-oxo-5- phenyl-1,3-dihydro- 1,4-benzodiazepin- 3-yl]pyrazolo[1,5- a]pyrimidine-3- carboxamide | F | 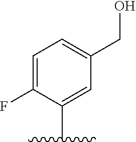 [SMILES: *c1cc(CO)ccc1F] | (400 MHz) δ 10.98 (s, 1H), 9.60 (d, J = 7.9 Hz, 1H), 9.42 (dd, J = 7.0, 1.7 Hz, 1H), 9.00 (dd, J = 4.3, 1.7 Hz, 1H), 7.62-7.56 (m, 1H), 7.55-7.38 (m, 8H), 7.35- 7.27 (m, 1H), 7.20-7.12 (m, 2H), 5.53 (d, J = 8.0 Hz, 1H), 5.29 (t, J = 5.8 Hz, 1H), 4.51 (d, J = 5.7 Hz, 2H). | 539.2 [M + H]⁺ |
| 11 | 2-(2-Fluoro-4- hydroxyphenyl)-N- [(3S)-2-oxo-5- phenyl-1,3-dihydro- 1,4-benzodiazepin- 3-yl]pyrazolo[1,5- a]pyrimidine-3- carboxamide | H | 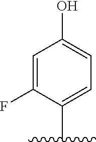 [SMILES: *c1ccc(O)cc1F] | (400 MHz) δ 11.00 (s, 1H), 10.12 (s, 1H), 9.60 (d, J = 8.1 Hz, 1H), 9.38 (dd, J = 7.0, 1.7 Hz, 1H), 8.96 (dd, J = 4.3, 1.7 Hz, 1H), 7.65 (ddd, J = 8.5, 7.0, 1.7 Hz, 1H), 7.55-7.22 (m, 10H), 6.65 (dd, J = 8.4, 2.3 Hz, 1H), 6.57 (dd, J = 11.8, 2.3 Hz, 1H), 5.46 (d, J = 8.1 Hz, 1H). | 505.4 [M − H]⁻ |
| 12 | 2-(2-Fluoro-4- hydroxyphenyl)-N- [(3S)-9-fluoro-2- oxo-5-phenyl-1,3- dihydro-1,4- benzodiazepin-3- yl]pyrazolo[1,5- a]pyrimidine-3- carboxamide | F | 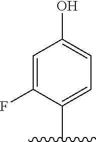 [SMILES: *c1ccc(O)cc1F] | (400 MHz) δ 10.96 (s, 1H), 10.11 (s, 1H), 9.61 (d, J = 8.0 Hz, 1H), 9.38 (dd, J = 7.0, 1.7 Hz, 1H), 8.96 (dd, J = 4.3, 1.7 Hz, 1H), 7.64-7.56 (m, 1H), 7.56-7.42 (m, 5H), 7.38 (dd, J = 7.0, 4.3 Hz, 1H), 7.35-7.29 (m, 2H), 7.18-7.13 (m, 1H), 6.66 (dd, J = 8.4, 2.3 Hz, 1H), | 525.4 [M + H]⁺ |
| | | | | 6.58 (dd, J = 11.8, 2.3 Hz, 1H), | |
| | | | | 5.53 (d, J = 8.0 Hz, 1H). | |
| 13 | 2-(1-Ethylpyrazol- 4-yl)-N-[(3S)-9- fluoro-2-oxo-5- phenyl-1,3-dihydro- 1,4-benzodiazepin- 3-yl]pyrazolo[1,5- a]pyrimidine-3- carboxamide | F | 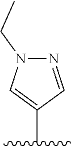 [SMILES: *c1cnn(CC)c1] | (400 MHz) δ 11.00 (s, 1H), 10.01 (d, J = 7.8 Hz, 1H), 9.33 (dd, J = 6.9, 1.7 Hz, 1H), 8.89 (dd, J = 4.3, 1.6 Hz, 1H), 8.77 (d, J = 0.4 Hz, 1H), 8.18 (d, J = 0.5 Hz, 1H), 7.63 (ddd, J = 10.4, 8.2, 1.4 Hz, 1H), 7.57-7.50 (m, 3H), 7.50-7.43 (m, 2H), 7.38- 7.30 (m, 2H), 7.22 (d, J = 8.0 Hz, | 509.2 [M + H]⁺ |
| | | | | 1H), 5.67 (d, J = 7.8 Hz, | |
| | | | | 1H), 4.19 (q, J = 7.3 Hz, 2H), | |
| | | | | 1.37 (t, J = 7.3 Hz, 3H). | |
| 14 | 2-[2-Fluoro-4-(2- hydroxy-2- methylpropyl)phenyl]- N-[(3S)-9-fluoro- 2-oxo-5-phenyl-1,3- dihydro-1,4- benzodiazepin-3- yl]pyrazolo[1,5- a]pyrimidine-3- carboxamide | F | 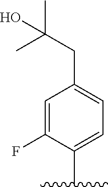 [SMILES: *c1ccc(CC(C)(C)O)cc1F] | (400 MHz) δ 10.98 (s, 1H), 9.63 (d, J = 8.0 Hz, 1H), 9.42 (dd, J = 7.0, 1.7 Hz, 1H), 8.99 (dd, J = 4.3, 1.7 Hz, 1H), 7.60 (ddd, J = 10.4, 8.2, 1.4 Hz, 1H), 7.56- 7.37 (m, 7H), 7.32 (td, J = 8.1, 5.0 Hz, 1H), 7.16 (dt, J = 7.9, 1.1 Hz, 1H), 7.12-7.05 (m, 2H), 5.55 (d, J = 8.0 Hz, 1H), 4.44 (s, 1H), 2.71 (s, 2H), 1.10 (d, J = 1.3 Hz, 6H). | 581.2 [M + H]⁺ |
| 15 | 2-[2-Fluoro-4-(2- hydroxy-2- methylpropyl)phenyl]- N-[(3S)-2-oxo-5- phenyl-1,3-dihydro- 1,4-benzodiazepin- 3-yl]pyrazolo[1,5- a]pyrimidine-3- carboxamide | H | 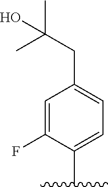 [SMILES: *c1ccc(CC(C)(C)O)cc1F] | (400 MHz) δ 11.02 (s, 1H), 9.61 (d, J = 8.1 Hz, 1H), 9.42 (dd, J = 7.0, 1.7 Hz, 1H), 8.99 (dd, J = 4.3, 1.7 Hz, 1H), 7.65 (ddd, J = 8.1, 7.1, 1.7 Hz, 1H), 7.56-7.38 (m, 7H), 7.36-7.30 (m, 2H), 7.26 (ddd, J = 8.1, 7.1, 1.1 Hz, 1H), 7.14-7.04 (m, 2H), 5.47 (d, J = 8.1 Hz, 1H), 4.44 (s, 1H), 2.71 (s, 2H), 1.10 (d, J = 1.2 Hz, 6H). | 563.2 [M + H]⁺ |
| 16 | 2-[2-Fluoro-4-(1- hydroxyethyl)phenyl]- N-[(3S)-2-oxo-5- phenyl-1,3-dihydro- 1,4-benzodiazepin- 3-yl]pyrazolo[1,5- a]pyrimidine-3- carboxamide | H | 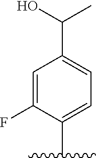 [SMILES: *c1ccc(C(C)O)cc1F] | (400 MHz) δ 11.01 (s, 1H), 9.59 (d, J = 8.1 Hz, 1H), 9.42 (dd, J = 7.0, 1.7 Hz, 1H), 8.99 (dd, J = 4.3, 1.7 Hz, 1H), 7.64 (ddd, J = 8.7, 7.1, 1.8 Hz, 1H), 7.56-7.37 (m, 7H), 7.31 (ddd, J = 7.8, 3.0, 1.4 Hz, 2H), 7.28-7.14 (m, 3H), 5.45 (d, J = 8.0 Hz, 1H), 5.35 (d, J = 4.4 Hz, 1H), 4.82- 4.72 (m, 1H), 1.34 (d, J = | 535.4 [M + H]⁺ |
| | | | | 6.4 Hz, 3H). | |
| 17 | 2-[2-Fluoro-4-(1- hydroxyethyl)pheny 1]-N-[(3S)-9-fluoro- 2-oxo-5-phenyl-1,3- dihydro-1,4- benzodiazepin-3- yl]pyrazolo[1,5- a]pyrimidine-3- carboxamide | F | 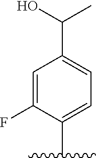 [SMILES: *c1ccc(C(C)O)cc1F] | (400 MHz) δ 10.97 (s, 1H), 9.61 (d, J = 7.9 Hz, 1H), 9.42 (dd, J = 7.0, 1.7 Hz, 1H), 8.99 (dd, J = 4.3, 1.7 Hz, 1H), 7.59 (ddd, J = 10.3, 8.2, 1.4 Hz, 1H), 7.57- 7.37 (m, 7H), 7.31 (td, J = 8.1, 5.1 Hz, 1H), 7.26-7.10 (m, 3H), 5.53 (d, J = 8.0 Hz, 1H), 5.35 (d, J = 4.5 Hz, 1H), 4.83- 4.72 (m, 1H), 1.35 (d, J = | 553.3 [M + H]⁺ |
| | | | | 6.5 Hz, 3H). | |
| 18 | 2-[2-Fluoro-3- (hydroxymethyl) phenyl]-N-[(3S)-9- fluoro-2-oxo-5- phenyl-1,3-dihydro- 1,4-benzodiazepin- 3-yl]pyrazolo[1,5- | F | 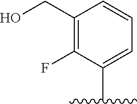 [SMILES: *c1cccc(CO)c1F] | (400 MHz) δ 10.96 (s, 1H), 9.61 (d, J = 8.0 Hz, 1H), 9.42 (dd, J = 7.0, 1.7 Hz, 1H), 9.00 (dd, J = 4.3, 1.7 Hz, 1H), 7.62-7.35 (m, 9H), 7.34-7.21 (m, 2H), 7.17- 7.11 (m, 1H), 5.53 (d, J = 8.0 Hz, 1H), 5.31 (t, J = 5.8 Hz, | 539.5 [M + H]⁺ |
| | a]pyrimidine-3- | | | 1H), 4.56 (d, J = 5.8 Hz, 2H). | |
| | carboxamide | | | | |
| 19 | 2-[2-Fluoro-4-(2- hydroxypropan-2- yl)phenyl]-N-[(3S)- 9-fluoro-2-oxo-5- phenyl-1,3-dihydro- 1,4-benzodiazepin- 3-yl]pyrazolo[1,5- a]pyrimidine-3- carboxamide | F | 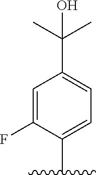 [SMILES: *c1ccc(C(C)(C)O)cc1F] | (700 MHz) δ 11.00 (s, 1H), 9.60 (d, J = 8.0 Hz, 1H), 9.42 (dd, J = 7.0, 1.7 Hz, 1H), 8.99 (dd, J = 4.2, 1.7 Hz, 1H), 7.65 (ddd, J = 8.5, 7.3, 1.6 Hz, 1H), 7.54-7.50 (m, 1H), 7.49-7.43 (m, 5H), 7.41 (dd, J = 7.0, 4.2 Hz, 1H), 7.35-7.30 (m, 3H), 7.30-7.24 (m, 2H), 5.46 (d, J = 8.0 Hz, 1H), 5.20 (s, 1H), 1.45 (s, 6H). | 567.0 [M + H]⁺ |
| 20 | 2-[2-Fluoro-4-(2- hydroxypropan-2- yl)phenyl]-N-[(3S)- 2-oxo-5-phenyl-1,3- dihydro-1,4- benzodiazepin-3- yl]pyrazolo[1,5- a]pyrimidine-3- carboxamide | H | 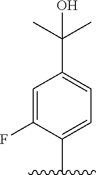 [SMILES: *c1ccc(C(C)(C)O)cc1F] | (700 MHz) δ 10.96 (s, 1H), 9.60 (d, J = 7.9 Hz, 1H), 9.41 (dd, J = 7.0, 1.6 Hz, 1H), 8.99 (dd, J = 4.3, 1.7 Hz, 1H), 7.59 (ddd, J = 10.2, 8.3, 1.3 Hz, 1H), 7.54- 7.48 (m, 3H), 7.48-7.42 (m, 3H), 7.40 (dd, J = 7.0, 4.3 Hz, 1H), 7.35-7.25 (m, 3H), 7.15 (dd, J = 7.8, 1.2 Hz, 1H), 5.53 (d, J = 7.9 Hz, 1H), 5.19 (s, 1H), 1.45 (s, 6H). | 549.0 [M + H]⁺ |
| 21 | 2-[2-Fluoro-4-(2- hydroxyethyl)phenyl]- N-[(3S)-2-oxo-5- phenyl-1,3-dihydro- 1,4-benzodiazepin- 3-yl]pyrazolo[1,5- a]pyrimidine-3- carboxamide | H | 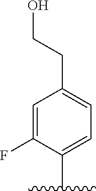 [SMILES: *c1ccc(CCO)cc1F] | (400 MHz,) δ 11.01 (s, 1H), 9.59 (d, J = 8.1 Hz, 1H), 9.41 (dd, J = 7.0, 1.7 Hz, 1H), 8.98 (dd, J = 4.3, 1.7 Hz, 1H), 7.68-7.60 (m, 1H), 7.55-7.36 (m, 7H), 7.34- 7.23 (m, 3H), 7.15-7.06 (m, 2H), 5.45 (d, J = 8.0 Hz, 1H), 4.72 (t, J = 5.1 Hz, 1H), 3.64 (td, J= 6.9, 5.1 Hz, 2H), 2.78 (t, J = 6.8 Hz, 2H). | 535.1 [M + H]⁺ |
| 22 | 2-[2-Fluoro-4-(2- hydroxyethyl)pheny 1]-N-[(3S)-9-fluoro- 2-oxo-5-phenyl-1,3- dihydro-1,4- benzodiazepin-3- yl]pyrazolo[1,5- a]pyrimidine-3- carboxamide | F | 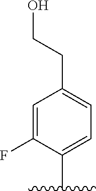 [SMILES: *c1ccc(CCO)cc1F] | (400 MHz) δ 10.98 (s, 1H), 9.61 (d, J = 7.9 Hz, 1H), 9.42 (dd, J = 7.0, 1.7 Hz, 1H), 8.99 (dd, J = 4.3, 1.7 Hz, 1H), 7.60 (ddd, J = 10.2, 8.3, 1.3 Hz, 1H), 7.58- 7.37 (m, 7H), 7.32 (td, J = 8.1, 5.0 Hz, 1H), 7.19-7.07 (m, 3H), 5.53 (d, J = 8.0 Hz, 1H), 4.73 (t, J = 5.1 Hz, 1H), 3.65 (td, J= 6.8, 5.1 Hz, 2H), 2.79 (t, J = 6.8 Hz, 2H). | 553.1 [M + H]⁺ |

### 23. 2-(4-Ethylsulfonyl-2-fluorophenyl)-N-[(3S)-9-fluoro-2-oxo-5-phenyl-1,3-dihydro-1,4-benzodiazepin-3-yl]pyrazolo[1,5-a]pyrimidine-3-carboxamide

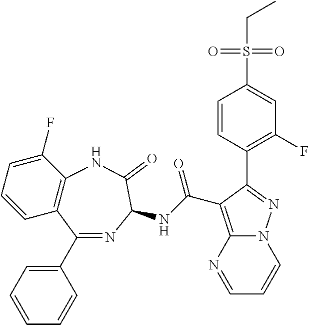 [SMILES: CCS(=O)(=O)c1ccc(-c2nn3cccnc3c2C(=O)N[C@H]2N=C(c3ccccc3)c3cccc(F)c3NC2=O)c(F)c1]

DIPEA (99 μL, 0.572 mmol) was added to a solution of intermediate 51 (100 mg, 0.286 mmol), (3S)-3-amino-9-fluoro-5-phenyl-1,3-dihydro-1,4-benzodiazepin-2-one (81 mg, 0.300 mmol) in DMF (3 mL) and the reaction mixture was stirred at rt for 3 min. HATU (120 mg, 0.315 mmol) was added and the reaction mixture was stirred at rt overnight. The reaction mixture was quenched with H₂O (15 mL), and the resultant precipitate collected by filtration, washing with water (3 ×15 mL). The precipitate was dissolved in EtOAc and the solvent removed under reduced pressure. Purification by flash chromatography (60-100% EtOAc in heptane) afforded an off-white solid (122 mg, 71%). ¹H NMR (400 MHz, DMSO-d₆) δ 10.98 (s, 1H), 9.59 (d, J=8.0 Hz, 1H), 9.46 (dd, J=6.8, 1.6 Hz, 1H), 9.03 (dd, J=4.0, 1.6 Hz, 1H), 7.86-7.79 (m, 3H), 7.61-7.56 (m, 1H), 7.55-7.43 (m, 6H), 7.34-7.28 (m, 1H), 7.16-7.14 (d, J=8.0 Hz, 1H), 5.53 (d, J=8.0 Hz, 1H), 3.41 (q, J=7.2 Hz, 2H), 1.14 (t, J=7.2 Hz, 3H). LRMS (ESI+) m/z 601.1 [M+H]⁺.

The following compounds were prepared by an analogous procedure to that described for the compound of Example 23.

TABLE 6
Examples compounds

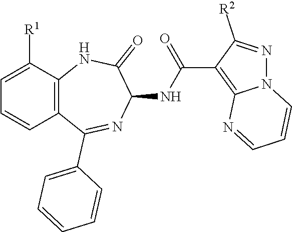

| | | | | ¹H NMR δ | LRMS m/z |
|---|---|---|---|---|---|
| Example | Name | R¹ | R² | (400 MHz, DMSO-d₆) | (APCI+) |
| 24 | N-[(3S)-9-Fluoro- 2-oxo-5-phenyl- 1,3-dihydro-1,4- benzodiazepin-3- yl]-2-(2-fluoro-4- sulfamoylphenyl)py razolo[1,5- a]pyrimidine-3- carboxamide | F | 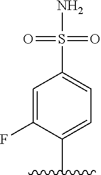 [SMILES: *c1ccc(S(N)(=O)=O)cc1F] | δ 10.98 (s, 1H), 9.59 (d, J = 8.0 Hz, 1H), 9.46-9.44 (m, 1H), 9.03-9.02 (m, 1H), 7.78- 7.72 (m, 2H), 7.65 (d, J = 9.6 Hz, 1H), 7.61-7.57 (m, 3H), 7.54-7.43 (m, 6H), 7.34-7.29 (m, 1H), 7.15 (d, J = 8.0 Hz, 1H), 5.54 (d, J = 7.6 Hz, 1H). | 588.1 [M + H]⁺ |
| 25 | 2-[4- (Dimethylsulfamoy 1)-2-fluorophenyl]- N-[(3S)-9-fluoro-2- oxo-5-phenyl-1,3- dihydro-1,4- benzodiazepin-3- yl]pyrazolo[1,5- a]pyrimidine-3- carboxamide | F | 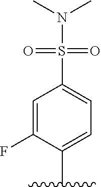 [SMILES: *c1ccc(S(=O)(=O)N(C)C)cc1F] | δ 10.98 (s, 1H), 9.60 (d, J = 8.0 Hz, 1H), 9.45 (dd, J = 6.8, 1.6 Hz, 1H), 9.03 (dd, J = 4.0, 1.6 Hz, 1H), 7.85-7.81 (m, 1H), 7.69-7.63 (m, 2H), 7.61- 7.56 (m, 1H), 7.55-7.43 (m, 6H), 7.34-7.28 (m, 1H), 7.15 (d, J = 7.6 Hz, 1H), 5.55 (d, J = 8.0 Hz, 1H), 2.69 (s, 6H). | 616.1 [M + H]⁺ |
| 26 | 2-(2-Fluoro-5- methylsulfonylphen yl)-N-[(3S)-9- fluoro-2-oxo-5- phenyl-1,3-dihydro- 1,4-benzodiazepin- 3-yl]pyrazolo[1,5- | F | 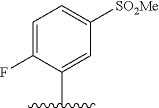 [SMILES: *c1cc(S(C)(=O)=O)ccc1F] | δ 10.97 (s, 1H), 9.60 (d, J = 7.6 Hz, 1H), 9.46 (dd, J = 6.8, 1.6 Hz, 1H), 9.04 (dd, J = 4.4, 2.0 Hz, 1H), 8.12-8.08 (m, 2H), 7.61-7.43 (m, 8H), 7.34- 7.28 (m, 1H), 7.15 (d, J = 7.6 Hz, 1H), 5.53 (d, J = 7.6 Hz, | 587.1 [M + H]⁺ |
| | a]pyrimidine-3- | | | 1H), 3.28 (s, 3H). | |
| | carboxamide | | | | |

### 27. 2-(4-Carbamoyl-2-fluorophenyl)-N-[(3S)-9-fluoro-2-oxo-5-phenyl-1,3-dihydro-1,4-benzodiazepin-3-yl]pyrazolo[1,5-a]pyrimidine-3-carboxamide

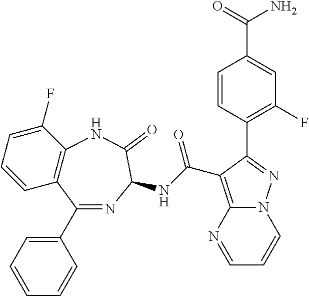 [SMILES: NC(=O)c1ccc(-c2nn3cccnc3c2C(=O)N[C@H]2N=C(c3ccccc3)c3cccc(F)c3NC2=O)c(F)c1]

A solution of intermediate 41 (110 mg, 0.34 mmol) and LiOH (1 M aq., 1.01 mL, 1.01 mmol) in THF/MeOH (3:1; 2 mL) was heated at 50° C. overnight. The solvents were removed under reduced pressure and the residue diluted with water (5 mL) and acidified with 1 M aq. HCl (1.34 mL). The resultant precipitate was filtered, washed with water and dried under reduced pressure to afford an orange solid (87 mg) which used without further purification. A mixture of 2-(4-carbamoyl-2-fluorophenyl)pyrazolo[1,5-a]pyrimidine-3-carboxylic acid and 2-(4-carboxy-2-fluorophenyl)pyrazolo[1,5-a]pyrimidine-3-carboxylic acid was obtained. (3S)-3-Amino-9-fluoro-5-phenyl-1,3-dihydro-1,4-benzodiazepin-2-one (79 mg, 0.29 mmol) was added to a portion of the crude residue (80 mg), followed by DMF (3 mL) and DIPEA (93 μL, 0.53 mmol) and the reaction mixture was stirred at rt for 3 min. HATU (122 mg, 0.32 mmol) was added and the reaction mixture was stirred at rt for 20 h. The reaction was quenched with water, and the resultant precipitate filtered, washing with water, then dissolved in (EtOH:CH₂Cl₂:NH₄OH; 50:8:1) and the volatiles removed under reduced pressure. Purification by flash chromatography [5-25% (EtOH:CH₂Cl₂:NH₄OH; 50:8:1) in CH₂Cl₂] afforded a white solid (20 mg). ¹H NMR (400 MHz, DMSO-d₆) δ 10.98 (s, 1H), 9.60 (d, J=8.0 Hz, 1H), 9.45 (dd, J=7.0, 1.7 Hz, 1H), 9.02 (dd, J =4.3, 1.7 Hz, 1H), 8.13 (s, 1H), 7.78 (dd, J=7.9, 1.6 Hz, 1H), 7.71 (dd, J=10.7, 1.6 Hz, 1H), 7.66-7.56 (m, 3H), 7.55-7.40 (m, 6H), 7.32 (td, J=8.1, 5.1 Hz, 1H), 7.15 (dt, J=7.9, 1.1 Hz, 1H), 5.53 (d, J=7.9 Hz, 1H). LRMS (APCI+) m/z 552.3 [M+H]⁺

The following compounds of the invention were prepared by chiral resolution from the denoted parent mixture of diastereomers by the method indicated and were isolated as a single stereoisomer of unknown absolute stereochemistry. The retention time for each resolved diastereomer under the indicated analytical conditions is denoted by t_{R}. The stereochemical configuration of these compounds has been designated as R* or S*, with the arbitrarily defined stereocenter marked by an asterisk.

TABLE 7
Resolution of Diastereomers

| | Preparative | | | |
|---|---|---|---|---|
| | Resolution | Analytical | Diastereo- | Diastereo- |
| Parent | Method | Method | mer 1 | mer 2 |
| Example | (Mobile Phase) | (Mobile Phase) | t_{R} (min) | tr (min) |
| 16 | SFC Method 1 | SFC Method 2A | 16-1 | 16-2 |
| | 50:50 EtOH:CO₂ | 60:40 MeOH:CO₂ | t_{R} = | t_{R} = |
| | (0.2% v/v NH₃) | (0.2% v/v NH₃) | 4.22 min | 6.09 min |
| 17 | SFC Method 1 | SFC Method 1A | 17-1 | 17-2 |
| | 50:50 EtOH:CO₂ | 50:50 EtOH:CO₂ | t_{R} = | t_{R} = |
| | (0.2% v/v NH₃) | (0.2% v/v NH₃) | 2.98 min | 4.10 min |

TABLE 8
Examples prepared as single diastereomers

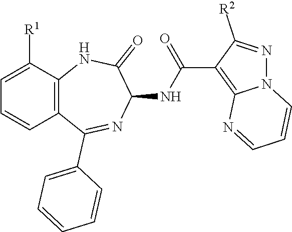

| | | | | ¹H NMR & | LCMS |
|---|---|---|---|---|---|
| Example | Name | R¹ | R² | (400 MHz, DMSO-d₆) | ESI+ m/z |
| 16-1 | 2-[2-Fluoro-4- [(1S*)-1- hydroxyethyl]phenyl]- N-[(3S)-2-oxo- 5-phenyl-1,3- dihydro-1,4- benzodiazepin-3- yl]pyrazolo[1,5- a]pyrimidine-3- carboxamide | H | 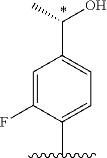 [SMILES: *c1ccc([C@H](C)O)cc1F] | 11.01 (s, 1H), 9.59 (d, J = 8.1 Hz, 1H), 9.42 (dd, J = 7.0, 1.7 Hz, 1H), 8.99 (dd, J= 4.3, 1.7 Hz, 1H), 7.67-7.61 (m, 1H), 7.54-7.38 (m, 7H), 7.35- 7.29 (m, 2H), 7.29-7.13 (m, 3H), 5.45 (d, J = 8.0 Hz, 1H), 5.35 (d, J = 4.4 Hz, 1H), 4.82- 4.73 (m, 1H), 1.34 (d, J = 6.5 Hz, 3H). | method A 535.5 [M + H]⁺ at 1.32 min |
| 16-2 | 2-[2-Fluoro-4- [(1R*)-1- hydroxyethyl]phenyl]- N-[(3S)-2-oxo- 5-phenyl-1,3- dihydro-1,4- benzodiazepin-3- yl]pyrazolo[1,5- a]pyrimidine-3- carboxamide | H | 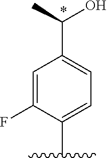 [SMILES: *c1ccc([C@@H](C)O)cc1F] | (11.01 (s, 1H), 9.59 (d, J = 8.1 Hz, 1H), 9.42 (dd, J = 7.0, 1.7 Hz, 1H), 8.99 (dd, J = 4.3, 1.7 Hz, 1H), 7.68-7.58 (m, 1H), 7.55-7.37 (m, 7H), 7.34- 7.28 (m, 2H), 7.28-7.12 (m, 3H), 5.44 (d, J = 8.0 Hz, 1H), 5.35 (d, J = 4.1 Hz, 1H), 4.81- 4.73 (m, 1H), 1.34 (d, J = 6.5 Hz, 3H). | method A 535.5 [M + H]⁺ at 1.32 min |
| 17-1 | 2-[2-Fluoro-4- [(1S*)-1- hydroxyethyl]phenyl]- N-[(3S)-9- fluoro-2-oxo-5- phenyl-1,3-dihydro- 1,4-benzodiazepin- 3-yl]pyrazolo[1,5- a]pyrimidine-3- carboxamide | F | 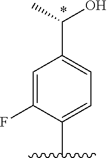 [SMILES: *c1ccc([C@H](C)O)cc1F] | 10.97 (s, 1H), 9.61 (d, J = 8.0 Hz, 1H), 9.42 (dd, J= 7.0, 1.7 Hz, 1H), 8.99 (dd, J = 4.3, 1.6 Hz, 1H), 7.63-7.56 (m, 1H), 7.55-7.38 (m, 7H), 7.31 (td, J = 8.1, 5.0 Hz, 1H), 7.25- 7.20 (m, 1H), 7.20-7.11 (m, 2H), 5.53 (d, J = 7.9 Hz, 1H), 5.35 (d, J = 4.5 Hz, 1H), 4.81- 4.72 (m, 1H), 1.35 (d, J = | method A 553.3 [M + H]⁺ at 1.46 min |
| | | | | 6.4 Hz, 3H). | |
| 17-2 | 2-[2-Fluoro-4- [(1R*)-1- hydroxyethyl]phenyl]- N-[(3S)-9- fluoro-2-oxo-5- phenyl-1,3-dihydro- 1,4-benzodiazepin- 3-yl]pyrazolo[1,5- a]pyrimidine-3- carboxamide | F | 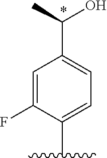 [SMILES: *c1ccc([C@@H](C)O)cc1F] | 10.97 (s, 1H), 9.61 (d, J = 7.9 Hz, 1H), 9.42 (dd, J = 7.0, 1.7 Hz, 1H), 8.99 (dd, J = 4.2, 1.7 Hz, 1H), 7.61-7.37 (m, 8H), 7.28 (td, J = 8.0, 5.0 Hz, 1H), 7.24-7.09 (m, 3H), 5.50 (d, J = 7.9 Hz, 1H), 5.35 (d, J = 4.4 Hz, 1H), 4.83-4.71 (m, 1H), 1.35 (d, J = 6.4 Hz, 3H). | method A 553.3 [M + H]⁺ at 1.46 min |

### 28. [3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl Aminoacetate Hydrochloride

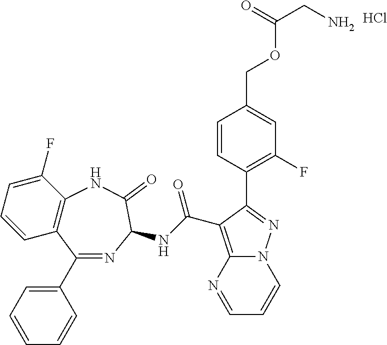 [SMILES: NCC(=O)OCc1ccc(-c2nn3cccnc3c2C(=O)N[C@H]2N=C(c3ccccc3)c3cccc(F)c3NC2=O)c(F)c1 ; Cl]

4 M HCl in dioxane (1.82 mL, 7.29 mmol) was added to a solution of intermediate 13A (169 mg, 0.24 mmol) in 1,4-dioxane (1.5 mL) at rt and stirred for 24 h. The mixture was diluted with MeOH and solvent removed under reduced pressure. Purification by flash chromatography [5-100% (EtOH:CH₂Cl₂:NH₄OH; 50:8:1) in CH₂Cl₂] afforded a white solid (95 mg). The resulting solid was dissolved in CH₂Cl₂ (2 mL), HCl (2 M in Et20; 1.8 mL, 3.64 mmol) added, and stirred at rt for 15 min. The mixture was evaporated under reduced pressure, the residual solid sonicated with acetone (5 mL), collected by filtration, washed with a small amount of acetone and dried at the pump to afford an off-white solid (103 mg, 67%). ¹H NMR (400 MHz, DMSO-d₆) δ 10.99 (s, 1H), 9.60 (d, J=7.9 Hz, 1H), 9.43 (dd, J=7.0, 1.7 Hz, 1H), 9.01 (dd, J=4.3, 1.7 Hz, 1H), 8.37 (s, 3H), 7.64-7.40 (m, 9H), 7.37-7.26 (m, 3H), 7.15 (d, J=8.0 Hz, 1H), 5.51 (d, J=7.8 Hz, 1H), 5.31 (s, 2H), 3.93 (q, J=5.6 Hz, 2H). LRMS (APCI+) m/z 596.2 [M+H]⁺.

The following compounds of the invention were prepared by an analogous procedure to that described for the compound of Example 28.

TABLE 9
Example compounds

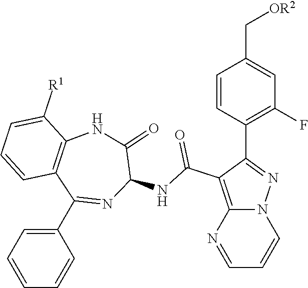

| | | | | | LRMS |
|---|---|---|---|---|---|
| | | | | ¹H NMR δ | m/z |
| Example | Name | R¹ | R² | (400 MHz, DMSO-d₆) | (APCI+) |
| 29 | 3-Fluoro-4-(3-{[(3S)- 2-oxo-5-phenyl-1,3- dihydro- 1,4-benzodiazepin-3- | H | 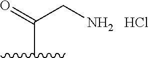 [SMILES: *C(=O)CN ; Cl] | 11.06 (s, 1H), 9.59 (d, J = 7.9 Hz, 1H), 9.44 (dd, J = 7.0, 1.7 Hz, 1H), 9.01 (dd, J = 4.3, 1.7 Hz, 1H), 8.42 (s, 3H), 7.69- | 578.2 [M + H]⁺ |
| | yl]carbamoyl} | | | 7.60 (m, 1H), 7.59-7.47 (m, | |
| | pyrazolo[1,5- | | | 2H), 7.49-7.39 (m, 5H), | |
| | a]pyrimidin-2- | | | 7.37-7.20 (m, 5H), 5.43 (d, | |
| | yl)phenyl]methyl | | | J = 7.9 Hz, 1H), 5.31 (s, 2H), | |
| | aminoacetate | | | 3.92 (q, J = 5.7 Hz, 2H). | |
| | hydrochloride | | | | |
| 30 | [3-Fluoro-4-(3- {[(3S)-2-oxo-5- phenyl-1,3-dihydro- 1,4-benzodiazepin-3- yl]carbamoyl} pyrazolo[1,5- a]pyrimidin-2- | H | 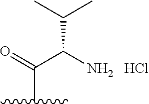 | 11.04 (s, 1H), 9.59 (d, J = 8.0 Hz, 1H), 9.43 (dd, J = 7.0, 1.7 Hz, 1H), 9.01 (dd, J = 4.3, 1.7 Hz, 1H), 8.52 (s, 3H), 7.68- 7.61 (m, 1H), 7.59-7.53 (m, 1H), 7.53-7.40 (m, 6H), 7.38-7.22 (m, 5H), 5.43 (d, | 620.3 [M + H]⁺ |
| | yl)phenyl]methyl | | | J = 8.0 Hz, 1H), 5.33 (q, 2H), | |
| | (2S)-2-amino-3- | | | 4.02-3.97 (m, 1H), 2.26- | |
| | methylbutanoate | | | 2.15 (m, 1H), 0.97 (dd, J = | |
| | hydrochloride | | | 10.6, 6.9 Hz, 6H). | |
| 31 | [3-Fluoro-4-(3- {[(3S)-9-fluoro-2- oxo-5-phenyl-1,3- dihydro-1,4- benzodiazepin-3- yl]carbamoyl}pyrazolo | F | 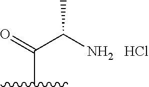 | 10.99 (s, 1H), 9.60 (d, J = 7.9 Hz, 1H), 9.43 (dd, J = 7.0, 1.7 Hz, 1H), 9.01 (dd, J = 4.3, 1.7 Hz, 1H), 8.48 (s, 3H), 7.65- 7.39 (m, 8H), 7.38-7.26 (m, 3H), 7.15 (d, J = 8.0 Hz, 1H), | 610.2 [M + H]⁺ |
| | [1,5-a]pyrimidin-2- | | | 5.51 (d, J = 7.8 Hz, 1H), 5.37- | |
| | yl)phenyl]methyl | | | 5.23 (m, 2H), 4.21 (s, 1H), | |
| | (2S)-2- | | | 1.44 (d, J = 7.2 Hz, 3H). | |
| | aminopropanoate | | | | |
| | hydrochloride | | | | |
| 32 | [3-Fluoro-4-(3- {[(3S)-9-fluoro-2- oxo-5-phenyl-1,3- dihydro-1,4- benzodiazepin-3- yl]carbamoyl}pyrazolo | F | 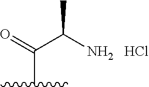 | 10.99 (s, 1H), 9.60 (d, J = 7.9 Hz, 1H), 9.43 (dd, J= 7.0, 1.7 Hz, 1H), 9.01 (dd, J= 4.3, 1.6 Hz, 1H), 8.50 (d, J = 5.5 Hz, 3H), 7.65-7.39 (m, 8H), 7.38-7.26 (m, 3H), 7.15 (d, | 610.1 [M + H]⁺ |
| | [1,5-a]pyrimidin-2- | | | J = 8.0 Hz, 1H), 5.51 (d, J = | |
| | yl)phenyl]methyl | | | 7.9 Hz, 1H), 5.37-5.23 (m, | |
| | (2R)-2- | | | 2H), 4.28-4.14 (m, 1H), | |
| | aminopropanoate | | | 1.45 (d, J = 7.2 Hz, 3H). | |
| | hydrochloride | | | | |
| 33 | [3-Fluoro-4-(3- {[(3S)-9-fluoro-2- oxo-5-phenyl-1,3- dihydro-1,4- benzodiazepin-3- yl]carbamoyl}pyrazolo [1,5-a]pyrimidin-2- | F | 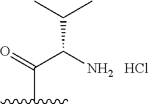 | 10.99 (s, 1H), 9.60 (d, J = 7.9 Hz, 1H), 9.43 (dd, J = 7.0, 1.7 Hz, 1H), 9.01 (dd, J= 4.3, 1.7 Hz, 1H), 8.49 (d, J = 5.5 Hz, 3H), 7.65-7.39 (m, 8H), 7.39-7.26 (m, 3H), 7.15 (d, J= 8.0 Hz, 1H), 5.51 (d, J = | 638.3 [M + H]⁺ |
| | yl)phenyl]methyl | | | 7.9 Hz, 1H), 5.32 (q, J = 12.8 Hz, | |
| | (2S)-2-amino-3- | | | 2H), 4.02 (t, J = 5.2 Hz, | |
| | methylbutanoate | | | 1H), 2.27-2.14 (m, 1H), | |
| | hydrochloride | | | 0.97 (dd, J = 10.1, 6.9 Hz, | |
| | | | | 6H) | |
| 34 | [3-Fluoro-4-(3- {[(3S)-9-fluoro-2- oxo-5-phenyl-1,3- dihydro-1,4- benzodiazepin-3- yl]carbamoyl}pyrazolo [1,5-a]pyrimidin-2- | F | 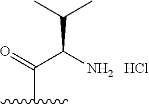 | 10.99 (s, 1H), 9.60 (d, J = 7.9 Hz, 1H), 9.43 (dd, J = 7.0, 1.7 Hz, 1H), 9.01 (dd, J= 4.3, 1.7 Hz, 1H), 8.49 (s, 3H), 7.67- 7.39 (m, 8H), 7.38-7.27 (m, 3H), 7.15 (d, J = 8.1 Hz, 1H), 5.51 (d, J = 7.9 Hz, 1H), 5.32 | 638.2 [M + H]⁺ |
| | yl)phenyl]methyl | | | (q, J = 12.8 Hz, 2H), 4.01 (d, | |
| | (2R)-2-amino-3- | | | J = 4.6 Hz, 1H), 2.29-2.14 | |
| | methylbutanoate | | | (m, 1H), 0.97 (dd, J = 10.0, | |
| | hydrochloride | | | 6.9 Hz, 6H). | |
| 35 | [3-Fluoro-4-(3- {[(3S)-9-fluoro-2- oxo-5-phenyl-1,3- dihydro-1,4- benzodiazepin-3- yl]carbamoyl}pyrazolo [1,5-a]pyrimidin-2- yl)phenyl]methyl (2S)-2-amino-4- | F | 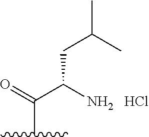 | 10.99 (s, 1H), 9.60 (d, J = 7.9 Hz, 1H), 9.43 (dd, J = 7.0, 1.7 Hz, 1H), 9.01 (dd, J= 4.3, 1.7 Hz, 1H), 8.61-8.43 (m, 3H), 7.67-7.39 (m, 10H), 7.38- 7.26 (m, 3H), 7.19-7.11 (m, 1H), 5.51 (d, J = 7.9 Hz, 1H), 5.42-5.18 (m, 2H), 4.09 (d, J = 6.1 Hz, 1H), 1.82-1.70 | 652.2 [M + H]⁺ |
| | methylpentanoate | | | (m, 1H), 1.70-1.57 (m, 2H), | |
| | hydrochloride | | | 0.89 (dd, J = 6.4, 2.7 Hz, 6H) | |
| 36 | [3-Fluoro-4-(3- {[(3S)-9-fluoro-2- oxo-5-phenyl-1,3- dihydro-1,4- benzodiazepin-3- yl]carbamoyl}pyrazolo [1,5-a]pyrimidin-2- yl)phenyl]methyl (2R)-2-amino-4- | F | 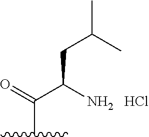 | 10.98 (s, 1H), 9.60 (d, J = 7.9 Hz, 1H), 9.43 (dd, J = 7.0, 1.7 Hz, 1H), 9.01 (dd, J = 4.3, 1.7 Hz, 1H), 8.50 (d, J = 5.1 Hz, 3H), 7.64-7.40 (m, 8H), 7.37-7.28 (m, 3H), 7.18- 7.12 (m, 1H), 5.51 (d, J = 7.9 Hz, 1H), 5.42-5.24 (m, 2H), 4.09 (d, J = 5.5 Hz, 1H), 1.82- | 652.6 [M + H]⁺ |
| | methylpentanoate | | | 1.59 (m, 3H), 0.89 (dd, J = | |
| | hydrochloride | | | 6.3, 2.6 Hz, 6H). | |

### 37. [3-Fluoro-4-[3-[[(3S)-9-fluoro-2-oxo-5-phenyl-1,3-dihydro-1,4-benzodiazepin-3-yl]carbamoyl]pyrazolo[1,5-a]pyrimidin-2-yl]phenyl]methyl Dihydrogen Phosphate

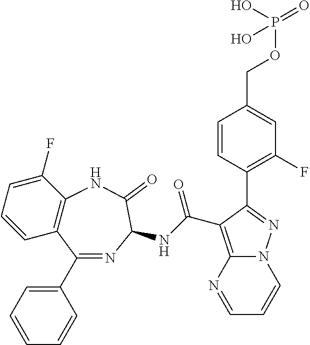 [SMILES: O=C(N[C@H]1N=C(c2ccccc2)c2cccc(F)c2NC1=O)c1c(-c2ccc(COP(=O)(O)O)cc2F)nn2cccnc12]

A reaction vessel was charged with 2-[2-fluoro-4-(hydroxymethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide (200 mg, 0.37 mmol), tetrabutylammonium hydrogensulfate (38 mg, 0.11 mmol) and phosphoenolpyruvate, monopotassium salt (345 mg, 1.67 mmol), diluted with DMF (2 mL) and heated at 100° C. for 124 h. The reaction was quenched with water, filtered, and the filtrate diluted with water (30 mL) and pH adjusted to 8 with sat. aq. NaHCO₃ and washed with CH₂Cl₂ (2×50 mL). The aqueous filtrate was evaporated to dryness under reduced pressure, the residue diluted with MeOH (50 mL), and filtered, washing with MeOH. The volatiles were removed under reduced pressure and the residue purified by preparative HPLC (method 2: MeCN/water with 0.2% v/v formic acid, 40-75% for 16 min) to afford a white solid (31 mg, 14%). ¹H NMR (400 MHz, DMSO-d₆) δ 10.96 (s, 1H), 9.64 (d, J=7.9 Hz, 1H), 9.40 (d, J=7.0 Hz, 1H), 8.96 (d, J=4.2 Hz, 1H), 7.44 (d, J=17.4 Hz, 7H), 7.31-6.79 (m, 5H), 5.33 (d, J=4.9 Hz, 1H), 4.74 (d, J=6.9 Hz, 2H). 31 P NMR (162 MHz, DMSO-d₆) δ 0.11. LCMS (method B) 619.5 [M+H]⁺ at 6.46 min.

### Example 38: Efficacy In Vitro

Compounds were subjected to RSV plaque reduction assays according to the following protocol. Plaque EC₅₀ and cell toxicity CC₅₀ values are a mean of at least two experiments and figures are rounded to whole units.

### Plaque Reduction Assay

Hep-G2 cells (ECACC, 85011430) were passaged in flasks and seeded in 24-well plates in DMEM containing antibiotics and supplemented with 10% FBS. During inoculation and subsequent incubation, cells were cultured in DMEM containing 2% FBS. 100 plaque forming unit/well of RSV (RSV A2 ECACC, 0709161v) was mixed with eight serial dilutions of compound. Subsequently, 100 μL of the virus/compound mixtures was added to confluent Hep-G2 cell monolayers. The cells and virus/compound mixtures were incubated at 37° C. in a humidified 5% CO₂ incubator for 2 h prior to removal of the inoculum and addition of 1 mL of overlay (DMEM containing 2% FBS and 0.8% CMC) containing compound dilutions. The cells and were incubated at 37° C. in a humidified 5% CO₂ incubator for 2 days.

Cells were washed with PBS before adding 75/25% v/v EtOH/MeOH, for 3 min. Fixative was removed and plates were washed with PBS. A pre-titrated amount of the primary antibody was added in 200 μL PBS/2% milk powder, and plates incubated for 90 min at 37° C. The plates were washed 3 times with PBS/0.05% Tween20 before addition of rabbit anti-goat horse radish peroxidase in 200 μL PBS/2% milk powder, and incubated for 1 h at 37° C. Following three wash steps with PBS/0.05% Tween20, 200 μL ready-to-use TrueBlue was added and plates were incubated at rt for 10-15 min before washing with water. After removal of water, plates were air-dried in the dark. Plates were scanned and analysed using the Immunospot S6 Macro analyser, which is equipped with BioSpot analysis software for counting immunostained plaques (virospots). Plaque counts were used to calculate % infection relative to the mean of the plaque count in the virus control wells for RSV. The EC₅₀ value was calculated as 50% reduction in signal, respectively, by interpolation of inhibition curves fitted with a 4-parameter nonlinear regression with a variable slope in Dotmatics. Plaque EC₅₀ and cell toxicity CC₅₀ values are a mean of at least two experiments and figures are rounded to whole units.

### Results

| | RSV A2 Plaque | Cell Cytotoxicity |
|---|---|---|
| Example | EC₅₀ (nM) | CC₅₀ (nM) |
| 1 | 18 | >25,000 |
| 2 | 40 | >25,000 |
| 3 | 66 | >25,000 |
| 4 | 37 | >25,000 |
| 5 | 66 | >25,000 |
| 6 | 49 | >25,000 |
| 7 | 9 | >25,000 |
| 8 | 13 | >25,000 (n = 1) |
| 9 | 60 | >25,000 |
| 10 | 25 | >20,246 |
| 11 | 47 | >25,000 |
| 12 | 16 | >25,000 |
| 13 | 58 | >25,000 |
| 14 | 20 | >25,000 |
| 15 | 32 | >25,000 |
| 16 | 7.6 | >25,000 (n = 1) |
| 17 | 6.9 | >25,000 (n = 1) |
| 18 | 44 | >25,000 |
| 19 | 8.5 | >25,000 |
| 20 | 8.7 | >25,000 (n = 1) |
| 21 | 42 | >25,000 |
| 22 | 25 | >25,000 |
| 23 | 3.0 | >25,000 (n = 1) |
| 24 | 49 | >25,000 |
| 25 | 1.2 | >25,000 (n = 1) |
| 26 | 61 | >25,000 |
| 27 | 49 | >25,000 |
| 16-1 | 8.2 | >2500 |
| 16-2 | 11 | >2500 |
| 17-1 | 5.5 | >2500 |
| 17-2 | 7.8 | >2500 |
| 29 | 49 | >25,000 |
| 30 | 44 | >25,000 |

### Example 39: In Vitro Pharmacokinetics

Compounds were subjected to the following assays to investigate liver microsomal stability and hepatocyte stability.

### Microsomal Incubation: Experimental Procedure

Pooled liver microsomes were purchased from a reputable commercial supplier and stored at −80° C. prior to use. Microsomes (final protein concentration 0.5 mg/mL), 0.1 M phosphate buffer pH 7.4 and test compound (final substrate concentration 1 μM; final DMSO concentration 0.25%) were pre-incubated at 37° C. prior to the addition of NADPH (final concentration 1 mM) to initiate the reaction. The final incubation volume was 50 μL. A control incubation was included for each compound tested where 0.1 M phosphate buffer pH 7.4 was added instead of NADPH (minus NADPH). Two control compounds were included with each species. All incubations were performed singularly for each test compound. Each compound was incubated for 0, 5, 15, 30 and 45 min. The control (minus NADPH) was incubated for 45 min only. The reactions were stopped by transferring incubate into acetonitrile at the appropriate time points, in a 1:3 ratio. The termination plates are centrifuged at 3,000 rpm for 20 min at 4° C. to precipitate the protein. Following protein precipitation, the sample supernatants were combined in cassettes of up to 4 compounds, internal standard added, and samples analysed by LC-MS/MS. From a plot of In peak area ratio (compound peak area/internal standard peak area) against time, the gradient of the line was determined. Subsequently, half-life (t_{1/2}) and intrinsic clearance (CLᵢₙₜ) were calculated. Compounds with low clearance (>80% remaining at 45 min) under the assay conditions are denoted as t_{1/2}>140 min.

### Results

| | Liver Microsomal Stability |
|---|---|
| Example | t_{1/2} (min); rat/dog/human |
| 1 | 41.4/635/329 |
| 2 | 57.4/53.2/117 |
| 3 | 209/107/>140 |
| 4 | 158/157/395 |
| 6 | 85.2/124/259 |
| 13 | 451/409/536 |
| 18 | 59/390/530 |
| 19 | 146/73.3/169 |

### Hepatocyte Incubation: Experimental Procedure

Cryopreserved pooled hepatocytes were purchased from a reputable commercial supplier and stored in liquid nitrogen prior to use. Williams E media supplemented with 2 mM L-glutamine and 25 mM HEPES and test compound (final substrate concentration 3 μM; final DMSO concentration 0.25%) are pre-incubated at 37° C. prior to the addition of a suspension of cryopreserved hepatocytes (final cell density 0.5 ×10⁶ viable cells/mL in Williams E media supplemented with 2 mM L-glutamine and 25 mM HEPES) to initiate the reaction. The final incubation volume is 500 μL. Two control compounds were included with each species, alongside appropriate vehicle control. The reactions were stopped by transferring 50 μL of incubate to 100 μL acetonitrile containing internal standard at the appropriate time points. Samples were removed at 6 time points (0, 5, 15, 30, 45 and 60 min) over the course of a 60 min experiment. The termination plates were centrifuged at 2500 rpm at 4° C. for 30 min to precipitate the protein.

Following protein precipitation, the sample supernatants were combined in cassettes of up to 4 compounds and analysed using generic LC-MS/MS conditions. From a plot of In peak area ratio (compound peak area/internal standard peak area) against time, the gradient of the line was determined. Subsequently, half-life (t_{1/2}) and intrinsic clearance (CLᵢₙₜ) were calculated. Compounds with low clearance (>80% remaining at 60 min) under the assay conditions are denoted as t_{1/2}>180 min.

### Results

| | Liver Hepatocyte Stability |
|---|---|
| Example | t_{1/2} (min); rat/dog/human |
| 1 | 83/157/333 |
| 4 | 130/84/227 |
| 7 | 5140/57/377 |
| 18 | 94/>180/>180 |

### Example 40: In Vivo Pharmacokinetics

The pharmacokinetics of compounds were studied in vivo in rats at doses of 1 mg/kg (IV) and 10 mg/kg (PO).

### Methods

Male rats [Sprague Dawley (SD)] surgically prepared with a jugular vein cannula were treated with experimental compounds via intravenous administration (IV; n=3; 1 mg/kg) or oral administration (PO; n=3; 10 mg/kg). Compounds were formulated as a solution in 40:60 dimethylacetamide:saline (IV administration) and a solution of 10% DMSO, 10% cremaphor in water (80%) (PO administration). Animals were observed for any overt clinical signs or symptoms. Serial blood samples were collected via the cannula at 0.02, 0.08, 0.25, 0.5, 1, 2, 4, 6, 8 and 24 h post IV dosing of compound, and at 0.08, 0.25, 0.5, 1, 2, 4, 6, 8 and 24 h post oral dosing of compound, and plasma was prepared by centrifugation and stored immediately at −80° C. Samples were subsequently thawed, prepared for analysis by protein precipitation with acetonitrile, and analysed by tandem LCMS using electrospray ionisation using a matrix-matched calibration curve. PK parameters were calculated from the resulting data.

### Results

| Example | 1 | 4 | 7 |
|---|---|---|---|
| PO AUCₗₐₛₜ | 3883 | 57054 | 24762 |
| (hr*ng/mL) | | | |
| Cl (mL/min/kg) | 11.5 | 1.9 | 13.1 |
| V_{d} (L/kg) | 0.75 | 0.49 | 1.4 |
| Cₘₐₓ (ng/mL) | 788 | 4793 | 2342 |
| IV t_{1/2} (h) | 1.5 | 4.4 | 1.6 |
| PO t_{1/2} (h) | 3.1 | 3.7 | 2.5 |
| F (%) | 25.6 | 54.6 | 188 |

### Example 41: Aqueous Formulation

The compound of Example 1 is formulated as a solution in 30% w/v captisol (i.e. sulfobutylether-beta-cyclodextrin) at pH4 according to the following procedure.

A carrier of 30% w/v captisol (i.e. sulfobutylether-beta-cyclodextrin) is prepared by weighing the required amount of captisol into a suitable vessel, adding approximately 80% of the final volume of water and magnetically stirring until a solution is formed. The carrier is then made up to volume with water.

An aqueous solution of a compound of Example 1 is prepared by weighing 175 mg of the compound into a suitable vessel and adding approximately 80% of the required volume of the carrier. Using an aqueous solution of hydrochloric acid, the pH is adjusted to pH2 and the resulting mixture is magnetically stirred until a solution is formed. The formulation is then made up to volume with carrier and the pH is adjusted to pH4 using an aqueous solution of sodium hydroxide.

### Example 42: Tablet Composition

Tablets, each weighing 0.15 g and containing 25 mg of a compound of the invention are manufactured as follows:

### Composition for 10,000 Tablets

| | |
|---|---|
| Compound of the invention | (250 g) |
| Lactose | (800 g) |
| Corn starch | (415 g) |
| Talc powder | (30 g) |
| Magnesium stearate | (5 g) |

The compound of the invention, lactose and half of the corn starch are mixed. The mixture is then forced through a sieve 0.5 mm mesh size. Corn starch (10 g) is suspended in warm water (90 mL). The resulting paste is used to granulate the powder. The granulate is dried and broken up into small fragments on a sieve of 1.4 mm mesh size. The remaining quantity of starch, talc and magnesium is added, carefully mixed and processed into tablets.

### Example 43: Injectable Formulation

| Compound of the invention | 200 | mg |
|---|---|---|
| Hydrochloric Acid Solution 0.1M or | 4.0 to 7.0 | |
| Sodium Hydroxide Solution 0.1M q.s. to pH | | |
| Sterile water q.s. to | 10 | mL |

The compound of the invention is dissolved in most of the water (35 ° C.-40° C.) and the pH adjusted to between 4.0 and 7.0 with the hydrochloric acid or the sodium hydroxide as appropriate. The batch is then made up to volume with water and filtered through a sterile micropore filter into a sterile 10 mL amber glass vial (type 1) and sealed with sterile closures and overseals.

### Example 44: Intramuscular Injection

| | | |
|---|---|---|
| Compound of the invention | 200 | mg |
| Benzyl Alcohol | 0.10 | g |
| Glycofurol 75 | 1.45 | g |
| Water for injection q.s to | 3.00 | mL |

The compound of the invention is dissolved in the glycofurol. The benzyl alcohol is then added and dissolved, and water added to 3 mL. The mixture is then filtered through a sterile micropore filter and sealed in sterile 3 mL glass vials (type 1).

### Example 45: Syrup Formulation

| | | |
|---|---|---|
| Compound of invention | 250 | mg |
| Sorbitol Solution | 1.50 | g |
| Glycerol | 2.00 | g |
| Sodium benzoate | 0.005 | g |
| Flavour | 0.0125 | mL |
| Purified Water q.s. to | 5.00 | mL |

The compound of the invention is dissolved in a mixture of the glycerol and most of the purified water. An aqueous solution of the sodium benzoate is then added to the solution, followed by addition of the sorbital solution and finally the flavour. The volume is made up with purified water and mixed well.

1. A compound which is a benzodiazepine of formula (I): 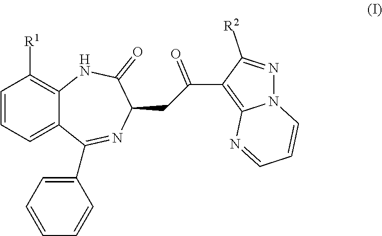
wherein:
R¹ is H or F;
R² is selected from: 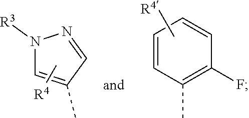
R³ is C₁-C₆ alkyl which is unsubstituted or substituted by CF₃, or is a monocyclic 4- to 6-membered heterocyclic group containing 1 or 2 heteroatoms selected from O, N and S;
R⁴ is H or a group selected from —X, -alk-X, —CONR⁶R⁷, C═NN R⁶R⁷, —SO₂R⁶ and —SO₂N R⁶R⁷;
R^{4′} is a group selected from —X, -alk-X, —CONR⁶R⁷, —C═NN R⁶R⁷, —SO₂R⁶ and —SO₂N R⁶R⁷;
X is —OH or a derivative of an OH group selected from α-amino carboxylic acid esters, carboxylic acid esters, carbonates, carbamates, ethers and phosphates;
alk is C₁-C₆ alkylene which is unsubstituted or substituted by C₃-C₆ cycloalkyl; and
R⁶ and R⁷ are each independently C₁-C₆ alkyl or C₃-C₆ cycloalkyl;
or a pharmaceutically acceptable salt thereof.

2. A compound according to claim 1 in which X is —OR⁸ in which R⁸ is H or a group selected from —C(O)—C(R¹⁰)(Y)—NR¹¹₂, —C(O)R⁹, —C(O)OR⁹, —C(O)NR¹²₂, —R⁹, —CH₂—OP(O)(OH)₂ and
—P(O)(OH)₂, wherein:
each R⁹ is independently C₁-C₆ alkyl which is unsubstituted or substituted;
each R¹⁰ is independently H or Me;
each R¹¹ is independently H, Me or a group —C(O)—CHY—NR¹⁰₂;
each R¹² is independently H or C₁-C₆ alkyl which is unsubstituted or substituted; and
Y is the side-chain of an α-amino carboxylic acid.

3. A compound according to claim 2 in which X is OH.

4. A compound according to claim 3 in which R^{4′} is a group selected from —X and -alk-X, wherein X is as defined in any one of claims 1 to 3.

5. A compound according to claim 3 in which R^{4′} is —CON R⁶R⁷, —C═NNR⁶R⁷, —SO₂R⁶ or —SO₂NR⁶R⁷, wherein R⁶ and R⁷ are as defined in claim 1.

6. A compound according to claim 3 in which R⁴ is H.

7. A compound according to claim 3 in which alk is selected from —CH₂, —CH₂CH₂—, —CH(CH₃)—, —C(CH₃)₂, —CH₂ CH(CH₃)—, —CH₂C(CH₃)₂— and cyclopropyl-substituted alkylene groups of formulae:

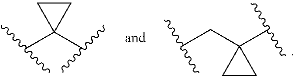 [SMILES: *C1(*)CC1 ; *CC1(*)CC1]

8. A compound which is selected from:
2-[2-Fluoro-4-(hydroxymethyl)phenyl]-N-[(3S)-9-fluoro-2-oxo-5-phenyl-1 ,3-dihydro-1,4-benzodiazepin-3-yl]pyrazolo[1,5-a]pyrimidine-3-carboxamide;
2-[2-Fluoro-4-(hydroxymethyl)phenyl]-N-[(3S)-2-oxo-5-phenyl-1,3-dihydro-1,4-benzodiazepin-3-yl]pyrazolo[1,5-a]pyrimidine-3-carboxamide;
N-[(3S)-9-fluoro-2-oxo-5-phenyl-1,3-dihydro-1,4-benzodiazepin-3-yl]-2-[1-(2,2,2-trifluoroethyl)pyrazol-4-yl]pyrazolo[1,5-a]pyrimidine-3-carboxamide;
N-[(3S)-9-fluoro-2-oxo-5-phenyl-1,3-dihydro-1,4-benzodiazepin-3-yl]-2-(1-propan-2-ylpyrazol-4-yl)pyrazolo[1,5-a]pyrimidine-3-carboxamide;
N-[(3S)-2-oxo-5-phenyl-1,3-dihydro-1,4-benzodiazepin-3-yl]-2-(1-propan-2-ylpyrazol-4-yl)pyrazolo[1,5-a]pyrimidine-3-carboxamide;
N-[(3S)-9-fluoro-2-oxo-5-phenyl-1,3-dihydro-1,4-benzodiazepin-3-yl]-2-[1-(oxan-4-yl)pyrazol-4-yl]pyrazolo[1,5-a]pyrimidine-3-carboxamide;
2-(2-Fluoro-4-methylsulfonylphenyl)-N-[(3S)-9-fluoro-2-oxo-5-phenyl-1,3-dihydro-1,4-benzodiazepin-3-yl]pyrazolo[1,5-a]pyrimidine-3-carboxamide;
2-(2-Fluoro-4-methylsulfonylphenyl)-N-[(3S)-2-oxo-5-phenyl-1,3-dihydro-1,4-benzodiazepin-3-yl]pyrazolo[1,5-a]pyrimidine-3-carboxamide;
2-[2-Fluoro-5-(hydroxymethyl)phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-5-(hydroxymethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide;
2-(2-Fluoro-4-hydroxyphenyl)-N-[(3S)-2-oxo-5-phenyl-1,3-dihydro-1,4-benzodiazepin-3-yl]pyrazolo[1,5-a]pyrimidine-3-carboxamide;
2-(2-Fluoro-4-hydroxyphenyl)-N-[(3S)-9-fluoro-2-oxo-5-phenyl-1,3-dihydro-1,4-benzodiazepin-3-yl]pyrazolo[1,5-a]pyrimidine-3-carboxamide;
2-(1-Ethylpyrazol-4-yl)-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-4-(2-hydroxy-2-methylpropyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-4-(2-hydroxy-2-methylpropyl)phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-4-(1-hydroxyethyl)phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-4-(1-hydroxyethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-3-(hydroxymethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-4-(2-hydroxypropan-2-yl)phenyl]-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-4-(2-hydroxypropan-2-yl)phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-4-(2-hydroxyethyl)phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-4-(2-hydroxyethyl)phenyl]-N-[(3S)-9-fluoro-2-oxo-5-phenyl-1,3-dihydro-1,4-benzodiazepin-3-yl]pyrazolo[1,5-a]pyrimidine-3-carboxamide;
2-(4-Ethylsulfonyl-2-fluorophenyl)-N-[(3S)-9-fluoro-2-oxo-5-phenyl-1,3-dihydro-1,4-benzodiazepin-3-yl]pyrazolo[1,5-a]pyrimidine-3-carboxamide;
N-[(3S)-9-Fluoro-2-oxo-5-phenyl-1,3-dihydro-1,4-benzodiazepin-3-yl]-2-(2-fluoro-4-sulfamoylphenyl)pyrazolo[1,5-a]pyrimidine-3-carboxamide;
2-[4-(Dimethylsulfamoyl)-2-fluorophenyl]-N-[(3S)-9-fluoro-2-oxo-5-phenyl-1,3-dihydro-1,4-benzodiazepin-3-yl]pyrazolo[1,5-a]pyrimidine-3-carboxamide;
2-(2-Fluoro-5-methylsulfonylphenyl)-N-[(3S)-9-fluoro-2-oxo-5-phenyl-1,3-dihydro-1,4-benzodiazepin-3-yl]pyrazolo[1,5-a]pyrimidine-3-carboxamide;
2-(4-Carbamoyl-2-fluorophenyl)-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-4-[(1S*)-1-hydroxyethyl]phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-4-[(1R*)-1-hydroxyethyl]phenyl]-N-[(3S)-2-oxo-5-phenyl-1,3-dihydro-1,4-benzodiazepin-3-yl]pyrazolo[1,5-a]pyrimidine-3-carboxamide;
2-[2-Fluoro-4-[(1S*)-1-hydroxyethyl]phenyl]-N-[(3S)-9-fluoro-2-oxo-5-phenyl-1,3-dihydro-1,4-benzodiazepin-3-yl]pyrazolo[1,5-a]pyrimidine-3-carboxamide;
2-[2-Fluoro-4-[(1R*)-1-hydroxyethyl]phenyl]-N-[(3S)-9-fluoro-2-oxo-5-phenyl-1,3-dihydro-1,4-benzodiazepin-3-yl]pyrazolo[1,5-a]pyrimidine-3-carboxamide;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl aminoacetate hydrochloride;
3-Fluoro-4-(3-{[(3S)-2-oxo-5-phenyl-1,3-dihydro-1,4-benzodiazepin-3-yl]carbamoyl}pyrazolo[1,5-a]pyrimidin-2-yl)phenyl]methyl aminoacetate hydrochloride;
[3-Fluoro-4-(3-{[(3S)-2-oxo-5-phenyl-1,3-dihydro-1,4-benzodiazepin-3-yl]carbamoyl}pyrazolo[1,5-a]pyrimidin-2-yl)phenyl]methyl (2S)-2-amino-3-methylbutanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2S)-2-aminopropanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2R)-2-aminopropanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2S)-2-amino-3-methylbutanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2R)-2-amino-3-methylbutanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2S)-2-amino-4-methylpentanoate hydrochloride;
[3-Fluoro-4-(3-{[(3S)-9-fluoro-2-oxo-5-phenyl-1,3-dihydro-1,4-benzodiazepin-3-yl]carbamoyl}pyrazolo[1,5-a]pyrimidin-2-yl)phenyl]methyl (2R)-2-amino-4-methylpentanoate hydrochloride;
[3-Fluoro-4-[3-[[(3S)-9-fluoro-2-oxo-5-phenyl-1,3-dihydro-1,4-benzodiazepin-3-yl]carbamoyl]pyrazolo[1,5-a]pyrimidin-2-yl]phenyl]methyl dihydrogen phosphate;and the pharmaceutically acceptable salts thereof.

9. A pharmaceutical composition which comprises a compound as defined in claim 1, and a pharmaceutically acceptable carrier or diluent.

10. A compound as defined in claim 1, for use in the treatment of the human or animal body by therapy.

11. A compound as defined in claim 1 for use in the treatment or prevention of an RSV infection.

12. Use of a compound as defined in claim 1, in the manufacture of a medicament for use in the treatment or prevention of an RSV infection

13. A method of treating a subject suffering from or susceptible to an RSV infection, which method comprises administering to said subject an effective amount of a compound as defined in claim 1.

14. A product containing:
(a) a compound as defined in claim 1; and
(b) one or more further therapeutic agents;
for simultaneous, separate or sequential use in the treatment of a subject suffering from or susceptible to an RSV infection.

15. A product according to claim 14, wherein the further therapeutic agent is:
a RSV nucleocapsid(N)-protein inhibitor;
(ii) a protein inhibitor, such as one that inhibits the phosphoprotein (P) protein and/or large (L) protein;
(iii) an anti-RSV monoclonal antibody, such as an F-protein antibody;
(iv) an immunomodulating toll-like receptor compound;
(v) a respiratory virus anti-viral, such as an anti-influenza and/or anti-rhinovirus compound; and/or
(vi) an anti-inflammatory compound.

16. A pharmaceutical composition which comprises (a) a compound as defined in claim 1, and (b) one or more therapeutic agents as defined in claim 15, together with a pharmaceutically acceptable carrier or diluent.

17. A process for producing a pharmaceutically acceptable salt as defined in claim 1, which process comprises treating a benzodiazepine derivative of formula (I) as defined in claim 1 with a suitable acid in a suitable solvent.

18. A process according to claim 17, wherein the acid is selected from hydrochloric acid, hydrobromic acid, hydroiodic acid, sulphuric acid, nitric acid, phosphoric acid, methanesulfonic acid, benzenesulphonic acid, formic acid, acetic acid, trifluoroacetic acid, propionic acid, oxalic acid, malonic acid, succinic acid, fumaric acid, maleic acid, lactic acid, malic acid, tartaric acid, citric acid, ethanesulfonic acid, aspartic acid and glutamic acid.
